(19) United States
(12) Patent Application Publication
(10) Pub. No.: US 2026/0344618 A1
(43) Pub. Date: Nov. 5, 2026

(54) METHODS, FORMULATIONS, AND DEVICES FOR GROOMING A PET HAVING A CURLY COAT

(71) Applicant: Quaranta Technology Solutions B.V., Rijswijk (NL)

(72) Inventors: Tiffany McKenzie, Maastricht (NL); Gabriel Alvarez, Maastricht (NL)

(21) Appl. No.: 19/447,174

(22) Filed: Jan. 13, 2026

Related U.S. Application Data
(60) Provisional application No. 63/744,435, filed on Jan. 13, 2025.

(51) Int. Cl. A01K 13/00; A46B 5/00; A46B 9/02; A46D 1/00; A61K 8/44; A61K 8/64; A61K 8/73; A61K 8/81; A61K 8/9789; A61Q 5/12

(52) U.S. Cl. CPC A01K 13/002; A46B 5/00; A46B 9/023; A46D 1/0253; A61K 8/44; A61K 8/64; A61K 8/737; A61K 8/8182; A61K 8/9789; A61Q 5/12; A46B 2200/104; A46B 2200/1093

(57) ABSTRACT

The present disclosure provides methods, conditioner formulations, and devices for grooming a pet (e.g., a dog) with a curly coat.

### PRIORITY CLAIM

This application claims priority to U.S. Provisional Patent Application Ser. No. 63/744,435, filed on Jan. 13, 2025, the entire contents of which are incorporated herein and relied upon.

### FIELD

The present disclosure provides methods, cream formulations, and devices for grooming a pet (e.g., a dog) with a curly coat (i.e., wavy or curly; not straight).

### BACKGROUND

Maintaining good health of a pet's curly coat is challenging. Products and methods currently available still leave curly coats tangled and/or matted after just a few days, prompting pet owners to frequently brush the curly coats. The positive effects from using currently available technologies are even shorter-lived when the pet's coat gets soaking wet, for example from exposure to heavy rain or from swimming. Currently available technologies, such as harsh shampoos and frequent brushing, often chemically and physically damage the coat's hair/fur, and undesirably attenuates the curls that pet owners seek to achieve and maintain. A need for reliable methods, products, and devices for grooming a pet having a curly coat still remains. The technologies of the present disclosure meet that need.

### SUMMARY

In one embodiment, the present disclosure provides a method of grooming a curly coat of a pet, the method comprising: shampooing the pet's coat; conditioning the pet's coat; brushing the pet's coat; drying the pet's coat until it remains damp; applying a curl cream to the pet's coat; gently blow drying the pet's coat; and waiting two weeks before shampooing, conditioning, or brushing the pet's coat.

In another embodiment, the present disclosure provides a curl cream or spray for establishing or maintaining a curly pet coat, the curl cream or spray comprising: a first conditioner; a second conditioner; a humectant; an emollient; a perfume; and a solvent.

In another embodiment, the present disclosure provides a curly coat brush for grooming a pet with a curly coat, the curly coat brush comprising: a base; a handle extending outwardly from the base; and a plurality of curly coat bristles, each curly coat bristle comprising: a base portion extending from the base, and a tip portion that extends at an obtuse angle from the base portion.

### BRIEF DESCRIPTION OF DRAWINGS

FIG. 1 shows a flow chart depicting a method for grooming a pet having a curly coat consistent with one embodiment of the present disclosure.

FIGS. 2A-2B show a flow chart depicting a method for grooming a pet having a curly coat consistent with a second embodiment of the present disclosure.

FIGS. 3A-3B show a flow chart depicting a method for grooming a pet having a curly coat consistent with a third embodiment of the present disclosure.

FIG. 4 shows a perspective view of a curly coat brush configured to groom a pet having a curly coat consistent with one embodiment of the present disclosure.

FIG. 5 shows a handle end view of the curly coat brush of FIG. 4.

FIG. 6 shows a right side view of the curly coat brush of FIG. 4, of which the left side is a mirror image.

FIG. 7 shows an enlarged side view of a portion of the curly coat brush of FIG. 4.

FIG. 8 shows a top view of the curly coat brush of FIG. 4.

FIG. 9 shows a bottom view of the curly coat brush of FIG. 4.

FIG. 10 shows a top end view of the curly coat brush of FIG. 4.

FIG. 11 shows a perspective view of a curly coat brush configured to groom a pet having a curly coat consistent with another embodiment of the present disclosure.

FIG. 12 shows an end view of the curly coat brush of FIG. 11.

FIG. 13 shows a right side view of the curly coat brush of FIG. 11, of which the left side is a mirror image.

FIG. 14 shows an enlarged side view of a portion of the curly coat brush of FIG. 11.

FIG. 15 shows a top view of the curly coat brush of FIG. 11.

FIG. 16 shows a bottom view of the curly coat brush of FIG. 11.

FIG. 17 shows a top end view of the curly coat brush of FIG. 11.

FIG. 18 shows a top perspective view of a curly coat brush consistent with another embodiment of the present disclosure.

FIG. 19 shows a right side view of the curly coat brush of FIG. 18.

FIG. 20 shows a front view of the curly coat brush of FIG. 18.

### DETAILED DESCRIPTION

In general, the present disclosure provides methods, cream formulations, and devices for grooming a pet (e.g., a dog) with a curly coat.

Employment of the methods, curly cream and spray formulations, and devices disclosed herein offer many advantages over currently available technologies. For example and without limitation, practicing the embodiments disclosed herein generally provide superior curled pet coats for longer durations than reliably possible using current state-of-the-art methods, formulations, and devices; and provides a more manageable and healthier curly pet coat compared to the reliable results available using current technologies.

### 1. Grooming Methods

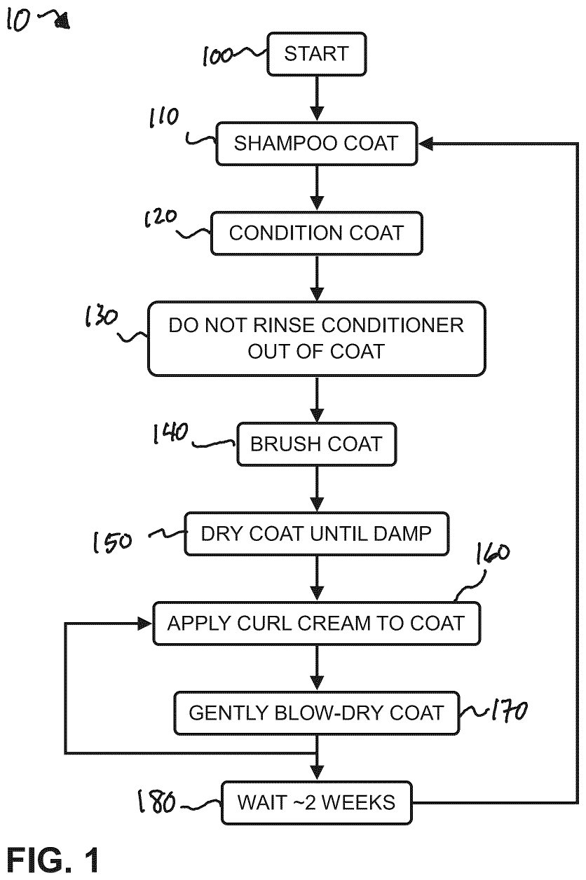

Referring now to FIGS. 1-3B, methods consistent with the present disclosure yield healthy curly coats for at least 2 weeks. Even when the pet's coat gets soaking wet during that time, methods of the present disclosure enable pet owners to maintain their pet's healthy curly coat more easily and quickly compared to currently available methods.

Referring now specifically to FIG. 1, a method 10 of the present disclosure generally begins 100 with a step of shampooing the pet's coat 110, followed by a step of conditioning the pet's coat 120. In step 130, the conditioner is not rinsed out of the pet's coat before the coat is brushed in step 140. Alternatively, the conditioner may be rinsed from the pet's coat before the coat is brushed in step 140, but the subsequent step 140 of brushing the coat may be less efficient or may yield undesirable results. In some embodiments, especially useful when the pet's coat is severely tangled, the step 140 of brushing the pet's coat includes: (i) applying curl cream to a section of the pet's damp (e.g., towel-dried only) coat, (ii) brushing that section of the pet's coat, and (iii) repeating steps (i)-(ii) until the entire pet's coat has been brushed. The coat is then dried until damp in step 150, at which time curl cream (e.g., a curl cream consistent with the present disclosure) is applied to the damp coat in step 160. The coat is then gently blow-dried in step 170. After waiting about 2 weeks (step 180), the method 10 can be repeated from the step 110 of shampooing the coat. During the 2-week period of step 180, the step 160 of applying curl cream to the coat and the step 170 of gently blow drying the coat may be repeated, for example if the pet's coat becomes soaking wet with water. In some embodiments, the method 10 does not include shampooing the pet's coat during the step 180 of waiting two weeks. In some embodiments, the method 10 does not include conditioning the pet's coat during the step 180 of waiting two weeks. In some embodiments, the method 10 does not include brushing the pet's coat during the step 180 of waiting two weeks. In some embodiments, the method 10 does not include shampooing, conditioning, or brushing the pet's coat during the step 180 of waiting two weeks. In some embodiments, the step 180 of waiting two weeks includes applying a curl spray formulation as disclosed herein to the pet's coat.

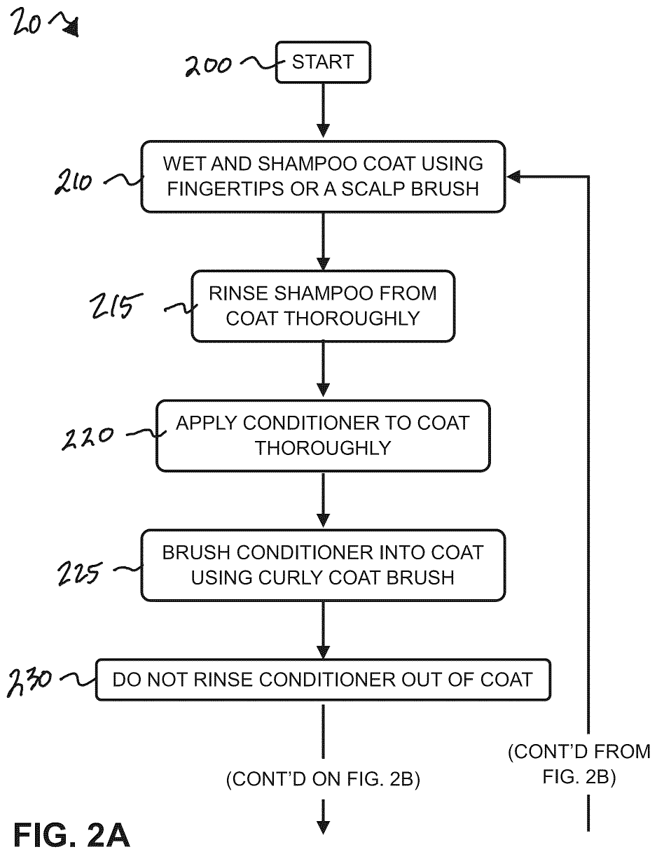

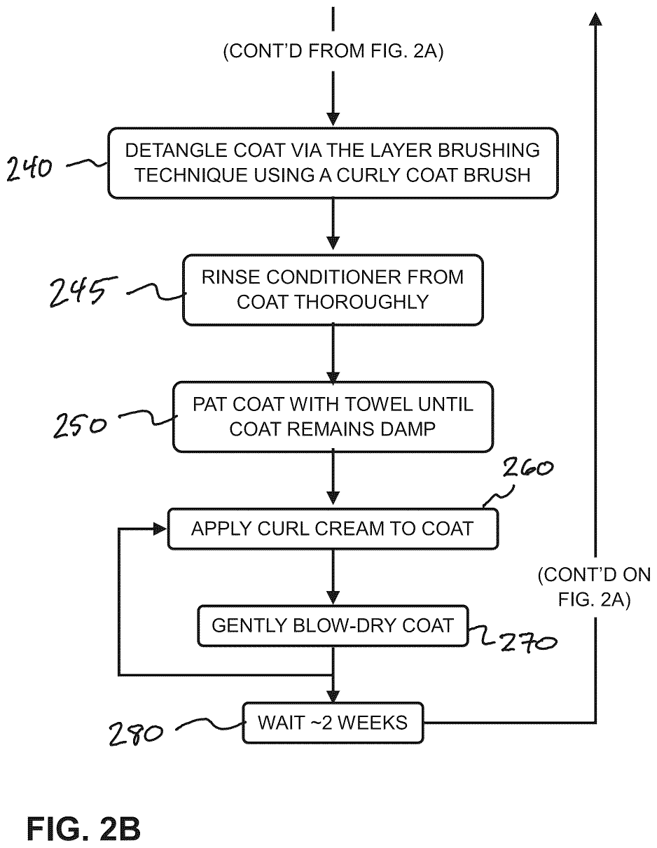

As shown representatively in FIGS. 2A-2B, a related method 20 begins 200 with a step 210 of wetting and shampooing the pet's coat using fingertips or a scalp brush. The shampoo is then rinsed thoroughly from the pet's coat (step 215) before conditioner is applied thoroughly to the coat (step 220), for example by brushing conditioner into the coat using a curly coat brush (step 225). The conditioner is not rinsed out of the coat (step 230) before the step 240 of detangling the coat by the layer brushing technique using a curly coat brush (e.g., a grooming brush consistent with the present disclosure). Alternatively, the conditioner may be rinsed from the pet's coat before the coat is detangled in step 240, but the subsequent step 240 of detangling the coat may be less efficient or may yield undesirable results. In some embodiments, especially useful when the pet's coat is severely tangled, the step 240 of detangling the pet's coat includes: (i) applying curl cream to a section of the pet's damp (e.g., towel-dried only) coat, (ii) brushing that section of the pet's coat, and (iii) repeating steps (i)-(ii) until the entire pet's coat has been brushed. The conditioner is then rinsed thoroughly from the pet's coat (step 245) and the coat is then patted with a towel until it remains damp (step 250). Curl cream (e.g., a curl cream consistent with the present disclosure) is applied to the damp coat in step 260, and the coat is then gently blow dried in step 270. After waiting about two weeks (step 280), the method 20 can be repeated from the step 210 of shampooing. If the pet's coat becomes soaking wet during the 2-week waiting period (step 280), the step 260 of applying curl cream to the coat and the step 270 of gently blow drying the coat may be repeated. In some embodiments, the method 20 does not include shampooing the pet's coat during the step 280 of waiting two weeks. In some embodiments, the method 20 does not include conditioning the pet's coat during the step 280 of waiting two weeks. In some embodiments, the method 20 does not include brushing the pet's coat during the step 280 of waiting two weeks. In some embodiments, the method 20 does not include shampooing, conditioning, or brushing the pet's coat during the step 280 of waiting two weeks. In some embodiments, the step 280 of waiting two weeks includes applying a curl spray formulation as disclosed herein to the pet's coat.

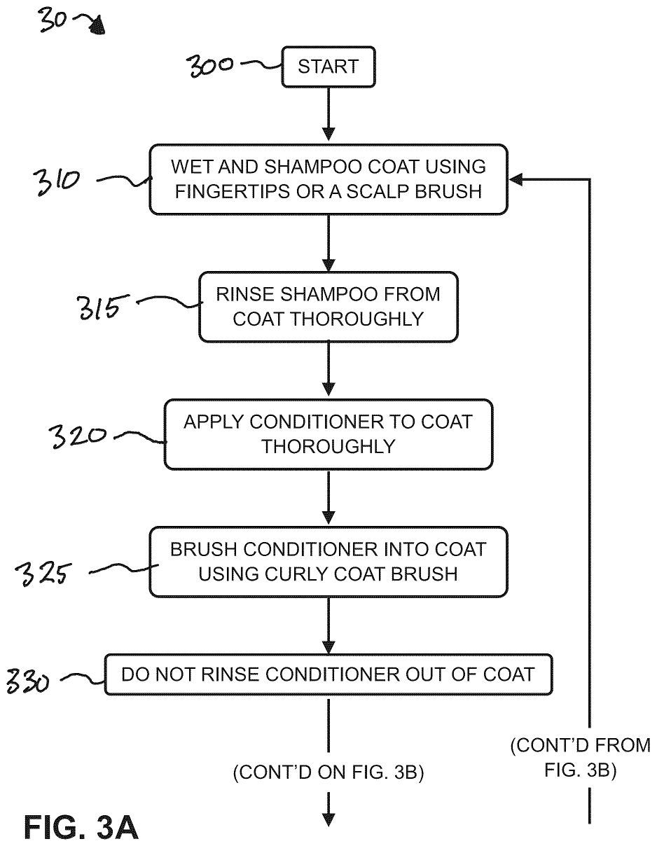

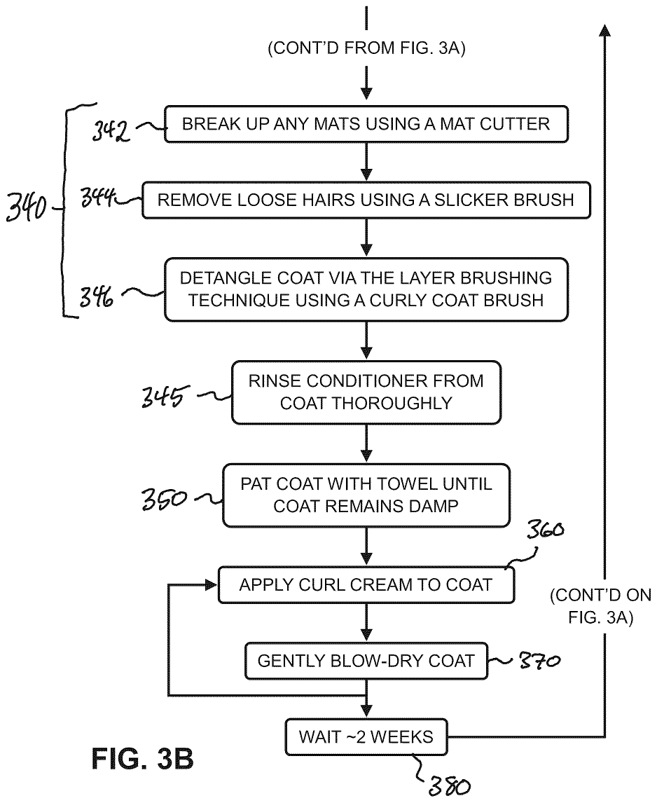

As shown representatively in FIGS. 3A-3B, a related method 30 begins 300 with a step 310 of wetting and shampooing the pet's coat using fingertips or a scalp brush. The shampoo is then rinsed thoroughly from the pet's coat (step 315) before conditioner is applied thoroughly to the coat (step 320), for example by brushing conditioner into the coat using a curly coat brush (step 325). The conditioner is not rinsed out of the coat (step 330) before the step 340 of brushing the coat is performed. Alternatively, the conditioner may be rinsed from the pet's coat before the coat is brushed in step 340, but the subsequent step 340 of brushing the coat may be less efficient or may yield undesirable results. The step 340 of brushing the coat in this method 30 includes a step 342 of breaking up any mats in the coat using a mat cutter device followed by a step 344 of removing any loose hairs using a slicker brush, and a step 346 of detangling the coat by the layer brushing technique using a curly coat brush (e.g., a grooming brush consistent with the present disclosure). In some embodiments, especially useful when the pet's coat is severely tangled, the step 340 of brushing the pet's coat is performed section-by-section and further includes: (i) a step of applying curl cream to a section of the pet's damp (e.g., towel-dried only) coat before (ii) steps 342-346 are performed on that section; then (iii) repeating steps (i)-(ii) section-by-section until the entire pet's coat has been brushed. The conditioner is then rinsed thoroughly from the pet's coat (step 345) and the coat is then patted with a towel until it remains damp (step 350). Curl cream (e.g., a curl cream consistent with the present disclosure) is applied to the damp coat in step 360, and the coat is then gently blow dried in step 370. After waiting about two weeks (step 380), the method 30 can be repeated from the step 310 of wetting and shampooing. If the pet's coat becomes soaking wet during the 2-week waiting period (step 380), the step 360 of applying curl cream to the coat and the step 370 of gently blow drying the coat may be repeated. In some embodiments, the method 30 does not include shampooing the pet's coat during the step 380 of waiting two weeks. In some embodiments, the method 30 does not include conditioning the pet's coat during the step 380 of waiting two weeks. In some embodiments, the method 30 does not include brushing the pet's coat during the step 380 of waiting two weeks. In some embodiments, the method 30 does not include shampooing, conditioning, or brushing the pet's coat during the step 380 of waiting two weeks. In some embodiments, the step 380 of waiting two weeks includes applying a curl spray formulation as disclosed herein to the pet's coat.

In general, the steps 140,240,340 of brushing the pet's coat should be performed while conditioner is still present in the pet's coat. In other words, methods 10-30 of the present disclosure provide superior results when the step 130,230,330 of not rinsing conditioner out of the pet's coat is included. However, the methods 10-30 of the present disclosure may be performed without the step 130,230,330 of not rinsing conditioner out of the pet's coat before the step 140,240,340 of brushing the pet's coat if necessary. In such alternative embodiments, an additional step of applying another type of lubricant may be included after a step of rinsing the conditioner out of the pet's coat and before the step 140,240,340 of brushing the pet's coat.

In general, the methods 10-30 of the present disclosure provide superior results when the pet's coat is treated with shampoo (e.g., steps 110,210-215,310-315) before the pet's coat is brushed.

In general, the methods 10-30 of the present disclosure provide superior results (e.g., yields fewer mats and tangles) when the step 160,260,360 of applying curl cream is performed on a pet's coat that is damp rather than dry.

The layer brushing technique referred to in steps 240 and 346, also referred to as the pat and pull technique, generally includes parting the pet's coat using a comb or fingers, patting the coat at the parted area using a slicker brush or a curly coat brush, pulling the brush through the top layer of fur/hair in the direction of its growth while lifting the brush away from the pet to avoid scratching the pet's skin, releasing a small portion of the parted hair into the parted valley, and repeating the steps of patting and pulling the brush and releasing a small portion of the parted hair until the pet's entire coat has been treated.

In any method 10-30 disclosed herein, the step 170,270,370 of blow drying the coat may include a first sub-step of using a blow dryer (with or without a diffuser attachment) set on medium heat and moved about the pet's entire coat at a distance of 20-30 cm (8-12 inches); and a second sub-step of using a blow dryer (with or without a diffuser attachment) set on low heat and moved about the pet's entire coat at a distance of 5-10 cm, taking care not to enable blown air to contact the same area of the coat for more than 5 seconds.

Any method 10-30 disclosed herein may additionally include a step of applying a drying aid (e.g., a “fast dry” spray) or adding dry shampoo powder to the pet's coat roots before the step 170,270,370 of blow drying the coat. In such embodiments, the pet's coat may dry more efficiently and/or may appear more voluminous after the step 170,270,370 of blow drying is complete.

Any method 10-30 disclosed herein may additionally include a step of applying a drying aid (e.g., a “fast dry” spray) or adding dry shampoo powder to the pet's coat roots after the step 170,270,370 of blow drying the coat. In such embodiments, the pet's coat may dry more efficiently and/or may appear more voluminous after the step of applying a drying aid or adding dry shampoo powder is complete.

Any method 10-30 disclosed herein may combine the step 110,210,310 of shampooing the coat and the step 120,220,320 of conditioning the coat by, for example, using a two-in-one shampoo and conditioner product. However, the pet coat may undesirably build up with product, and the curl cream applied in step 160,260,360 may not absorb into the pet coat as efficiently when the shampooing and conditioning steps are combined. In some embodiments, the two-in-one shampoo and conditioner product is not rinsed from the pet's coat (step 130,230,330) before the subsequent step 140,340 of brushing the coat or the subsequent step 240 of detangling the coat. The two-in-one shampoo and conditioner may then optionally be rinsed from the pet's coat after the step 140,340 of brushing or the step 240 of detangling. In other embodiments, the two-in-one shampoo and conditioner product is rinsed from the pet's coat before the subsequent step 140,340 of brushing the coat or the subsequent step 240 of detangling the coat.

Any method 10-30 disclosed herein may omit the step 110,210,310 of shampooing the pet coat. However, the subsequent step 160,260,360 of applying the curl cream may result in less efficient adsorption/absorption of the curl cream, and curl cream and other product applied to the pet coat may undesirably build up.

Any method 10-30 disclosed herein may include a pretreatment step of generously applying a lubricant, such as coconut oil or curl cream, to the pet coat, and then brushing the pet coat, before the step 110,210,310 of shampooing the pet coat. The pretreatment step may be especially useful when the pet coat is in very bad condition (e.g., extremely dirty or very tangled). Optionally, the step 110 of shampooing the pet coat, or the steps 210-215/310-315 of shampooing and rinsing the pet coat, may be repeated to remove all of the lubricant before the step 120,220,320 of conditioning the coat.

In another embodiment consistent with the present disclosure, a method of treating a pet's coat shortly after it has been groomed (e.g., that removes or substantially straightens out the pet's curly coat) comprises: (a) shampooing the pet's coat, (b) thoroughly rinsing the shampoo from the pet's coat, (c) applying conditioner to the pet's coat, (d) allowing the conditioner to remain in the pet's coat for at least 5 minutes, (e) thereafter rinsing the conditioner from the pet's coat, (f) applying curl cream to the pet's coat, and (g) gently blow drying the pet's coat. While this method is unlikely to completely restore the curliness of the pet's coat immediately after the coat has been fluffed or blown out, it can hasten the process by which the pet's coat returns to a naturally curly appearance.

In any method 10-30 disclosed herein, the step 160,260,360 of applying curl cream to the pet's coat may include the sub-steps of: (i) dispensing a small dollop of curl cream into a user's hand and covering both palms and all fingers of the user's hands by rubbing them together; (ii) spreading the curl cream on a section of the pet's coat using a raking motion that directs the fur/hair away from the pet's body at a 90° angle; (iii) ensuring that the curl cream is applied all the way to the base of the pet's fur/hair throughout the section of the pet's coat; and (iv) repeating sub-steps (i)-(iii) until all sections of the pet's coat have been thoroughly treated with curl cream.

### 2. Curl Cream and Curl Spray Formulations

The present disclosure also provides curl cream and spray formulations useful to develop and maintain a pet's healthy curly coat. In some embodiments, a curl cream or spray formulation consistent with the present disclosure reduces and/or prevents matting of the pet's coat and strongly defines the coat's curl characteristics. In some embodiments, a curl cream or spray formulation consistent with the present disclosure reduces and/or prevents matting of the pet's coat and imparts soft, bouncy curls. In some embodiments, a curl cream or spray formulation consistent with the present disclosure reduces and/or prevents matting of the pet's coat for at least 2 weeks without additional grooming (e.g., shampooing or conditioning). In some embodiments, a curl cream or spray formulation consistent with the present disclosure reduces and/or prevents matting of the pet's coat for at least 2 weeks, strongly defines the coat's curl characteristics, and imparts soft, bouncy curls.

In general, curl cream formulations consistent with the present disclosure have a relatively high viscosity such that, after application to a pet's coat (e.g., by massaging the curl cream composition throughout the pet coat by hand), the curl cream composition remains adhered to the pet coat hairs. In some embodiments, curl cream formulations include relatively high amounts of a thickener (e.g., a gelling agent) like a carbomer (e.g., Carbomer 940) compared to a comparable curl spray formulation.

In contrast, curl spray formulations consistent with the present disclosure have a relatively low viscosity to enable application to the pet coat through a spray nozzle (e.g., a spray nozzle associated with a spray bottle) without clogging the nozzle. In some embodiments, curl spray formulations consistent with the present disclosure include relatively low amounts of non-aqueous (e.g., oily) components compared to a comparable curl cream formulation to, for example, inhibit separation of phases in the curl spray formulation.

### Conditioners

Curl cream and spray formulations consistent with the present disclosure include at least one conditioner. In some embodiments, the curl cream or spray formulation includes a single conditioner. In other embodiments, the curl cream or spray formulation includes two conditioners. In still other embodiments, the curl cream or spray formulation includes three conditioners. In some embodiments, the curl cream or spray formulation includes four conditioners. In other embodiments, the curl cream or spray formulation includes more than four conditioners.

Typically, curl cream and spray formulations consistent with the present disclosure include at least at least two conditioners selected from the group consisting of: linseed extract, marshmallow root extract, arginine (base), and glycerin. In some embodiments, the curl cream or spray formulation includes at least linseed extract, marshmallow root extract, and arginine (base) as conditioners. In some embodiments, the curl cream or spray formulation includes linseed extract, marshmallow root extract, and a third conditioner selected from the group consisting of: arginine (base), silk amino acids, and guar hydroxypropyltrimonium chloride. In some embodiments, the curl cream or spray formulation includes linseed extract, marshmallow root extract, and silk amino acids as conditioners. In some embodiments, the curl cream or spray formulation includes linseed extract, marshmallow root extract, silk amino acids, and guar hydroxypropyltrimonium chloride as conditioners.

The conditioners are present in a total amount of 3% v/v to 50% v/v, for example 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, 16% v/v, 17% v/v, 18% v/v, 19% v/v, 20% v/v, 21% v/v, 22% v/v, 23% v/v, 24% v/v, 25% v/v, 26% v/v, 27% v/v, 28% v/v, 29% v/v, 30% v/v, 31% v/v, 32% v/v, 33% v/v, 34% v/v, 35% v/v, 36% v/v, 37% v/v, 38% v/v, 39% v/v, 40% v/v, 41% v/V, 42% v/v, 43% v/v, 44% v/v, 45% v/v, 46% v/v, 47% v/v, 48% v/v, 49% v/v, or 50% v/v.

In some embodiments, the conditioners include linseed extract. In some embodiments, the conditioners include marshmallow (Althaea officinalis) root extract. In some embodiments, the conditioners include arginine (base). In some embodiments, the conditioners include silk amino acids. In some embodiments, the conditioners include guar hydroxypropyltrimonium chloride (also known as Guar Excell). In some embodiments, the conditioners include linseed extract, marshmallow root extract, and arginine (base). In some embodiments, the conditioners include linseed extract, marshmallow root extract, and silk amino acids. In some embodiments, the conditioners include linseed extract, marshmallow root extract, silk amino acids, and guar hydroxypropyltrimonium chloride.

When the formulation includes linseed extract, it may be present in an amount of 0.5% v/v to 5% v/v, for example 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, 2% v/v, 2.1% v/v, 2.2% v/v, 2.3% v/v, 2.4% v/v, 2.5% v/v, 2.6% v/v, 2.7% v/v, 2.8% v/v, 2.9% v/v, 3% v/v, 3.1% v/v, 3.2% v/v, 3.3% v/v, 3.4% v/v, 3.5% v/v, 3.6% v/v, 3.7% v/v, 3.8% v/v, 3.9% v/v, 4% v/v, 4.1% v/v, 4.2% v/v, 4.3% v/v, 4.4% v/v, 4.5% v/v, 4.6% v/v, 4.7% v/v, 4.8% v/v, 4.9% v/v, or 5% v/v.

When the formulation includes marshmallow root extract, it may be present in an amount of 0.01% v/v to 2% v/v, for example 0.01% v/v, 0.02% v/v, 0.03% v/v, 0.04% v/V, 0.05% v/v, 0.06% v/v, 0.07% v/v, 0.08% v/v, 0.09% v/v, 0.1% v/v, 0.15% v/v, 0.2% v/v, 0.25% v/v, 0.3% v/v, 0.35% v/v, 0.4% v/v, 0.45% v/v, 0.5% v/v, 0.55% v/v, 0.6% v/V, 0.65% v/v, 0.7% v/v, 0.75% v/v, 0.8% v/v, 0.85% v/v, 0.9% v/v, 0.95% v/v, 1% v/V, 1.05% v/v, 1.1% v/v, 1.15% v/v, 1.2% v/v, 1.25% v/v, 1.3% v/v, 1.35% v/v, 1.4% v/V, 1.45% v/v, 1.5% v/v, 1.55% v/v, 1.6% v/v, 1.65% v/v, 1.7% v/v, 1.75% v/v, 1.8% v/v, 1.85% v/v, 1.9% v/v, 1.95% v/v, or 2% v/v.

When the formulation includes arginine (base), it may be present in an amount of 0.05% v/v to 5% v/v, for example 0.05% v/v, 0.06% v/v, 0.07% v/v, 0.08% v/v, 0.09% v/v, 0.1% v/v, 0.15% v/v, 0.2% v/v, 0.25% v/v, 0.3% v/v, 0.35% v/v, 0.4% v/v, 0.45% v/V, 0.5% v/v, 0.5% v/v, 0.6% v/v, 0.7% v/v, 0.8% v/v, 0.9% v/v, 1% v/v, 1.1% v/v, 1.2% v/V, 1.3% v/v, 1.4% v/v, 1.5% v/v, 1.6% v/v, 1.7% v/v, 1.8% v/v, 1.9% v/v, 2% v/v, 2.1% v/V, 2.2% v/v, 2.3% v/v, 2.4% v/v, 2.5% v/v, 2.6% v/v, 2.7% v/v, 2.8% v/v, 2.9% v/v, 3% v/V, 3.1% v/v, 3.2% v/v, 3.3% v/v, 3.4% v/v, 3.5% v/v, 3.6% v/v, 3.7% v/v, 3.8% v/v, 3.9% v/V, 4% v/v, 4.1% v/v, 4.2% v/v, 4.3% v/v, 4.4% v/v, 4.5% v/v, 4.6% v/v, 4.7% v/v, 4.8% v/v, 4.9% v/v, or 5% v/v.

When the formulation includes silk amino acids, it may be present in an amount of 0.1% v/v to 2% v/v, for example 0.1% v/v, 0.15% v/v, 0.2% v/v, 0.25% v/v, 0.3% v/V, 0.33% v/v, 0.35% v/v, 0.4% v/v, 0.45% v/v, 0.5% v/v, 0.55% v/v, 0.6% v/v, 0.65% v/v, 0.7% v/v, 0.75% v/v, 0.8% v/v, 0.85% v/v, 0.9% v/v, 0.95% v/v, 1% v/v, 1.05% v/v, 1.1% v/v, 1.15% v/v, 1.2% v/v, 1.25% v/v, 1.3% v/v, 1.35% v/v, 1.4% v/v, 1.45% v/v, 1.5% v/V, 1.55% v/v, 1.6% v/v, 1.65% v/v, 1.7% v/v, 1.75% v/v, 1.8% v/v, 1.85% v/v, 1.9% v/V, 1.95% v/v, or 2% v/V.

When the formulation includes guar hydroxypropyltrimonium chloride, it may be present in an amount of 0.01% v/v to 4% v/v, for example 0.01% v/v, 0.02% v/v, 0.03% v/v, 0.04% v/v, 0.05% v/v, 0.06% v/v, 0.07% v/v, 0.08% v/v, 0.09% v/v, 0.1% v/v, 0.15% v/v, 0.2% v/v, 0.25% v/v, 0.3% v/v, 0.35% v/v, 0.4% v/v, 0.45% v/v, 0.5% v/v, 0.55% v/V, 0.6% v/v, 0.65% v/v, 0.7% v/v, 0.75% v/v, 0.8% v/v, 0.85% v/v, 0.9% v/v, 0.95% v/v, 1% v/v, 1.05% v/v, 1.1% v/v, 1.15% v/v, 1.2% v/v, 1.25% v/v, 1.3% v/v, 1.35% v/v, 1.4% v/V, 1.45% v/v, 1.5% v/v, 1.55% v/v, 1.6% v/v, 1.65% v/v, 1.7% v/v, 1.75% v/v, 1.8% v/V, 1.85% v/v, 1.9% v/v, 1.95% v/v, 2% v/v, 2.05% v/v, 2.1% v/v, 2.15% v/v, 2.2% v/V, 2.25% v/v, 2.3% v/v, 2.35% v/v, 2.4% v/v, 2.45% v/v, 2.5% v/v, 2.55% v/v, 2.6% v/V, 2.65% v/v, 2.7% v/v, 2.75% v/v, 2.8% v/v, 2.85% v/v, 2.9% v/v, 2.95% v/v, 3% v/V, 3.05% v/v, 3.1% v/v, 3.15% v/v, 3.2% v/v, 3.25% v/v, 3.3% v/v, 3.35% v/v, 3.4% v/V, 3.45% v/v, 3.5% v/v, 3.55% v/v, 3.6% v/v, 3.65% v/v, 3.7% v/v, 3.75% v/v, 3.8% v/V, 3.85% v/v, 3.9% v/v, 3.95% v/v, or 4% v/V.

When the formulation includes linseed extract and marshmallow root extract as conditioners, the ratio of linseed extract to marshmallow root extract may be 1:4 to 500:1, for example 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 205:1, 210:1, 215:1, 220:1, 225:1, 230:1, 235:1, 240:1, 245:1, 250:1, 255:1, 260:1, 265:1, 270:1, 275:1, 280:1, 285:1, 290:1, 295:1, 300:1, 305:1, 310:1, 315:1, 320:1, 325:1, 330:1, 335:1, 340:1, 345:1, 350:1, 355:1, 360:1, 365:1, 370:1, 375:1, 380:1, 385:1, 390:1, 395:1, 400:1, 405:1, 410:1, 415:1, 420:1, 425:1, 430:1, 435:1, 440:1, 445:1, 450:1, 455:1, 460:1, 465:1, 470:1, 475:1, 480:1, 485:1, 490:1, 495:1, or 500:1.

When the formulation includes linseed extract and arginine (base) as conditioners, the ratio of linseed extract to arginine (base) may be 1:10 to 100:1, for example 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1.

When the formulation includes linseed extract and silk amino acids as conditioners, the ratio of linseed extract to silk amino acids may be 1:4 to 50:1, for example 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, or 50:1.

When the formulation includes linseed extract and guar hydroxypropyltrimonium chloride as conditioners, the ratio of linseed extract to guar hydroxypropyltrimonium chloride may be 1:8 to 50:1, for example 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, or 50:1.

### Humectants

In some embodiments, the curl cream includes a humectant. When included, the humectant may be present in an amount of 0.001% v/v to 1% v/v, for example 0.001% v/v, 0.002% v/v, 0.003% v/v, 0.004% v/v, 0.005% v/v, 0.006% v/v, 0.007% v/v, 0.008% v/v, 0.009% v/v, 0.01% v/v, 0.011% v/v, 0.012% v/v, 0.013% v/v, 0.014% v/v, 0.015% v/v, 0.016% v/v, 0.017% v/v, 0.018% v/v, 0.019% v/v, 0.02% v/v, 0.021% v/v, 0.022% v/v, 0.023% v/v, 0.024% v/v, 0.025% v/v, 0.026% v/v, 0.027% v/v, 0.028% v/v, 0.029% v/v, 0.03% v/v, 0.031% v/v, 0.032% v/v, 0.033% v/v, 0.034% v/v, 0.035% v/v, 0.036% v/v, 0.037% v/v, 0.038% v/v, 0.039% v/v, 0.04% v/v, 0.041% v/v, 0.042% v/v, 0.043% v/v, 0.044% v/v, 0.045% v/v, 0.046% v/v, 0.047% v/v, 0.048% v/v, 0.049% v/v, 0.05% v/v, 0.055% v/v, 0.06% v/v, 0.065% v/v, 0.07% v/v, 0.075% v/v, 0.08% v/v, 0.085% v/V, 0.09% v/v, 0.095% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.21% v/v, 0.22% v/v, 0.23% v/v, 0.24% v/v, 0.25% v/v, 0.26% v/v, 0.27% v/v, 0.28% v/v, 0.29% v/v, 0.3% v/v, 0.31% v/v, 0.32% v/v, 0.33% v/v, 0.34% v/v, 0.35% v/v, 0.36% v/v, 0.37% v/v, 0.38% v/V, 0.39% v/v, 0.4% v/v, 0.41% v/v, 0.42% v/v, 0.43% v/v, 0.44% v/v, 0.45% v/v, 0.46% v/V, 0.47% v/v, 0.48% v/v, 0.49% v/v, 0.5% v/v, 0.51% v/v, 0.52% v/v, 0.53% v/v, 0.54% v/v, 0.55% v/v, 0.56% v/v, 0.57% v/v, 0.58% v/v, 0.59% v/v, 0.6% v/v, 0.61% v/v, 0.62% v/V, 0.63% v/v, 0.64% v/v, 0.65% v/v, 0.66% v/v, 0.67% v/v, 0.68% v/v, 0.69% v/v, 0.7% v/v, 0.71% v/v, 0.72% v/v, 0.73% v/v, 0.74% v/v, 0.75% v/v, 0.76% v/v, 0.77% v/v, 0.78% v/v, 0.79% v/v, 0.8% v/v, 0.81% v/v, 0.82% v/v, 0.83% v/v, 0.84% v/v, 0.85% v/v, 0.86% v/v, 0.87% v/v, 0.88% v/v, 0.89% v/v, 0.9% v/v, 0.91% v/v, 0.92% v/v, 0.93% v/v, 0.94% v/v, 0.95% v/v, 0.96% v/v, 0.97% v/v, 0.98% v/v, 0.99% v/v, or 1% v/v.

In some embodiments, the humectant is aloe barbadensis leaf juice. In other embodiments, the humectant is another humectant safe for use on pet fur, safe for use on pet hair, and safe when it contacts pet skin.

### Emulsifiers

In some embodiments, the curl cream includes one or more emulsifiers. When included, the emulsifiers may be present in a total amount of 0.1% v/v to 15% v/v, for example 0.1% v/v, 0.15% v/v, 0.2% v/v, 0.25% v/v, 0.3% v/v, 0.33% v/v, 0.35% v/V, 0.4% v/v, 0.45% v/v, 0.5% v/v, 0.55% v/v, 0.6% v/v, 0.65% v/v, 0.7% v/v, 0.75% v/V, 0.8% v/v, 0.85% v/v, 0.9% v/v, 0.95% v/v, 1% v/v, 1.05% v/v, 1.1% v/v, 1.15% v/v, 1.2% v/v, 1.25% v/v, 1.3% v/v, 1.35% v/v, 1.4% v/v, 1.45% v/v, 1.5% v/v, 1.55% v/v, 1.6% v/V, 1.65% v/v, 1.7% v/v, 1.75% v/v, 1.8% v/v, 1.85% v/v, 1.9% v/v, 1.95% v/v, 2% v/v, 2% v/v, 2.1% v/v, 2.2% v/v, 2.3% v/v, 2.4% v/v, 2.5% v/v, 2.6% v/v, 2.7% v/v, 2.8% v/v, 2.9% v/v, 3% v/v, 3.1% v/v, 3.2% v/v, 3.3% v/v, 3.4% v/v, 3.5% v/v, 3.6% v/v, 3.7% v/v, 3.8% v/v, 3.9% v/v, 4% v/v, 4.1% v/v, 4.2% v/v, 4.3% v/v, 4.4% v/v, 4.5% v/v, 4.6% v/v, 4.7% v/v, 4.8% v/v, 4.9% v/v, 5% v/v, 5.1% v/v, 5.2% v/v, 5.3% v/v, 5.4% v/v, 5.5% v/v, 5.6% v/v, 5.7% v/v, 5.8% v/v, 5.9% v/v, 6% v/v, 6.1% v/v, 6.2% v/v, 6.3% v/v, 6.4% v/v, 6.5% v/v, 6.6% v/v, 6.7% v/v, 6.8% v/v, 6.9% v/v, 7% v/v, 7.1% v/v, 7.2% v/v, 7.3% v/v, 7.4% v/v, 7.5% v/v, 7.6% v/v, 7.7% v/v, 7.8% v/v, 7.9% v/v, 8% v/v, 8.1% v/v, 8.2% v/v, 8.3% v/v, 8.4% v/v, 8.5% v/v, 8.6% v/v, 8.7% v/v, 8.8% v/v, 8.9% v/v, 9% v/v, 9.1% v/v, 9.2% v/v, 9.3% v/v, 9.4% v/v, 9.5% v/v, 9.6% v/v, 9.7% v/v, 9.8% v/v, 9.9% v/v, 10% v/V, 10.1% v/v, 10.2% v/v, 10.3% v/v, 10.4% v/v, 10.5% v/v, 10.6% v/v, 10.7% v/v, 10.8% v/v, 10.9% v/v, 11% v/v, 11.1% v/v, 11.2% v/v, 11.3% v/v, 11.4% v/v, 11.5% v/v, 11.6% v/v, 11.7% v/v, 11.8% v/v, 11.9% v/v, 12% v/v, 12.1% v/v, 12.2% v/v, 12.3% v/v, 12.4% v/v, 12.5% v/v, 12.6% v/v, 12.7% v/v, 12.8% v/v, 12.9% v/v, 13% v/v, 13.1% v/v, 13.2% v/v, 13.3% v/v, 13.4% v/v, 13.5% v/v, 13.6% v/v, 13.7% v/v, 13.8% v/v, 13.9% v/v, 14% v/v, 14.1% v/v, 14.2% v/v, 14.3% v/v, 14.4% v/v, 14.5% v/v, 14.6% v/v, 14.7% v/V, 14.8% v/v, 14.9% v/v, or 15% v/v.

In some embodiments, the emulsifier is cetearyl alcohol/behentrimonium methosulfate. In other embodiments, the emulsifier is glyceryl stearate.

In embodiments including two or more emulsifier species, each emulsifier species may be present in an individual amount (independent of each other) of 0.1% v/v to 15% v/v, for example 0.1% v/v, 0.15% v/v, 0.2% v/v, 0.25% v/v, 0.3% v/v, 0.33% v/V, 0.35% v/v, 0.4% v/v, 0.45% v/v, 0.5% v/v, 0.55% v/v, 0.6% v/v, 0.65% v/v, 0.7% v/V, 0.75% v/v, 0.8% v/v, 0.85% v/v, 0.9% v/v, 0.95% v/v, 1% v/v, 1.05% v/v, 1.1% v/V, 1.15% v/v, 1.2% v/v, 1.25% v/v, 1.3% v/v, 1.35% v/v, 1.4% v/v, 1.45% v/v, 1.5% v/V, 1.55% v/v, 1.6% v/v, 1.65% v/v, 1.7% v/v, 1.75% v/v, 1.8% v/v, 1.85% v/v, 1.9% v/V, 1.95% v/v, 2% v/v, 2% v/v, 2.1% v/v, 2.2% v/v, 2.3% v/v, 2.4% v/v, 2.5% v/v, 2.6% v/V, 2.7% v/v, 2.8% v/v, 2.9% v/v, 3% v/v, 3.1% v/v, 3.2% v/v, 3.3% v/v, 3.4% v/v, 3.5% v/V, 3.6% v/v, 3.7% v/v, 3.8% v/v, 3.9% v/v, 4% v/v, 4.1% v/v, 4.2% v/v, 4.3% v/v, 4.4% v/V, 4.5% v/v, 4.6% v/v, 4.7% v/v, 4.8% v/v, 4.9% v/v, 5% v/v, 5.1% v/v, 5.2% v/v, 5.3% v/V, 5.4% v/v, 5.5% v/v, 5.6% v/v, 5.7% v/v, 5.8% v/v, 5.9% v/v, 6% v/v, 6.1% v/v, 6.2% v/V, 6.3% v/v, 6.4% v/v, 6.5% v/v, 6.6% v/v, 6.7% v/v, 6.8% v/v, 6.9% v/v, 7% v/v, 7.1% v/V, 7.2% v/v, 7.3% v/v, 7.4% v/v, 7.5% v/v, 7.6% v/v, 7.7% v/v, 7.8% v/v, 7.9% v/v, 8% v/v, 8.1% v/v, 8.2% v/v, 8.3% v/v, 8.4% v/v, 8.5% v/v, 8.6% v/v, 8.7% v/v, 8.8% v/v, 8.9% v/v, 9% v/v, 9.1% v/v, 9.2% v/v, 9.3% v/v, 9.4% v/v, 9.5% v/v, 9.6% v/v, 9.7% v/v, 9.8% v/V, 9.9% v/v, 10% v/v, 10.1% v/v, 10.2% v/v, 10.3% v/v, 10.4% v/v, 10.5% v/v, 10.6% v/V, 10.7% v/v, 10.8% v/v, 10.9% v/v, 11% v/v, 11.1% v/v, 11.2% v/v, 11.3% v/v, 11.4% v/V, 11.5% v/v, 11.6% v/v, 11.7% v/v, 11.8% v/v, 11.9% v/v, 12% v/v, 12.1% v/v, 12.2% v/V, 12.3% v/v, 12.4% v/v, 12.5% v/v, 12.6% v/v, 12.7% v/v, 12.8% v/v, 12.9% v/v, 13% v/V, 13.1% v/v, 13.2% v/v, 13.3% v/v, 13.4% v/v, 13.5% v/v, 13.6% v/v, 13.7% v/v, 13.8% v/v, 13.9% v/v, 14% v/v, 14.1% v/v, 14.2% v/v, 14.3% v/v, 14.4% v/v, 14.5% v/v, 14.6% v/v, 14.7% v/v, 14.8% v/v, 14.9% v/v, or 15% v/v. In some such embodiments, the emulsifiers include a combination of cetearyl alcohol/behentrimonium methosulfate and glyceryl stearate.

### Moisturizers

In some embodiments, the curl cream or spray formulation includes a moisturizer, for example to increase ease of applying the curl cream to pet fur or pet hair. When included, the moisturizer may be present in an amount of 0.1% to 5%, for example 0.1% v/v, 0.15% v/v, 0.2% v/v, 0.25% v/v, 0.3% v/v, 0.35% v/v, 0.4% v/V, 0.45% v/v, 0.5% v/v, 0.55% v/v, 0.6% v/v, 0.65% v/v, 0.7% v/v, 0.75% v/v, 0.8% v/V, 0.85% v/v, 0.9% v/v, 0.95% v/v, 1% v/v, 1.05% v/v, 1.1% v/v, 1.15% v/v, 1.2% v/V, 1.25% v/v, 1.3% v/v, 1.35% v/v, 1.4% v/v, 1.45% v/v, 1.5% v/v, 1.55% v/v, 1.6% v/V, 1.65% v/v, 1.7% v/v, 1.75% v/v, 1.8% v/v, 1.85% v/v, 1.9% v/v, 1.95% v/v, 2% v/V, 2.05% v/v, 2.1% v/v, 2.15% v/v, 2.2% v/v, 2.25% v/v, 2.3% v/v, 2.35% v/v, 2.4% v/V, 2.45% v/v, 2.5% v/v, 2.55% v/v, 2.6% v/v, 2.65% v/v, 2.7% v/v, 2.75% v/v, 2.8% v/V, 2.85% v/v, 2.9% v/v, 2.95% v/v, 3% v/v, 3.05% v/v, 3.1% v/v, 3.15% v/v, 3.2% v/V, 3.25% v/v, 3.3% v/v, 3.35% v/v, 3.4% v/v, 3.45% v/v, 3.5% v/v, 3.55% v/v, 3.6% v/V, 3.65% v/v, 3.7% v/v, 3.75% v/v, 3.8% v/v, 3.85% v/v, 3.9% v/v, 3.95% v/v, 4% v/V, 4.05% v/v, 4.1% v/v, 4.15% v/v, 4.2% v/v, 4.25% v/v, 4.3% v/v, 4.35% v/v, 4.4% v/v, 4.45% v/v, 4.5% v/v, 4.55% v/v, 4.6% v/v, 4.65% v/v, 4.7% v/v, 4.75% v/v, 4.8% v/v, 4.85% v/v, 4.9% v/v, 4.95% v/v, or 5% v/v.

In some embodiments, the moisturizer is slippery elm (Umus flulva) bark extract. In other embodiments, the moisturizer is another moisturizer safe for use on pet fur, safe for use on pet hair, and safe when it contacts pet skin.

### Film Forming Agents

In some embodiments, the curl cream or spray formulation includes a film forming agent, for example to increase the duration of effectiveness, to increase resistance to humidity, to increase softness of the pet hair/fur, and/or to increase protection from environmental factors. When included, the film forming agent may be present in an amount of 0.1% to 5%, for example for example 0.1% v/v, 0.15% v/v, 0.2% v/v, 0.25% v/v, 0.3% v/v, 0.35% v/v, 0.4% v/v, 0.45% v/v, 0.5% v/v, 0.55% v/v, 0.6% v/V, 0.65% v/v, 0.7% v/v, 0.75% v/v, 0.8% v/v, 0.85% v/v, 0.9% v/v, 0.95% v/v, 1% v/V, 1.05% v/v, 1.1% v/v, 1.15% v/v, 1.2% v/v, 1.25% v/v, 1.3% v/v, 1.35% v/v, 1.4% v/V, 1.45% v/v, 1.5% v/v, 1.55% v/v, 1.6% v/v, 1.65% v/v, 1.7% v/v, 1.75% v/v, 1.8% v/V, 1.85% v/v, 1.9% v/v, 1.95% v/v, 2% v/v, 2.05% v/v, 2.1% v/v, 2.15% v/v, 2.2% v/V, 2.25% v/v, 2.3% v/v, 2.35% v/v, 2.4% v/v, 2.45% v/v, 2.5% v/v, 2.55% v/v, 2.6% v/V, 2.65% v/v, 2.7% v/v, 2.75% v/v, 2.8% v/v, 2.85% v/v, 2.9% v/v, 2.95% v/v, 3% v/V, 3.05% v/v, 3.1% v/v, 3.15% v/v, 3.2% v/v, 3.25% v/v, 3.3% v/v, 3.35% v/v, 3.4% v/V, 3.45% v/v, 3.5% v/v, 3.55% v/v, 3.6% v/v, 3.65% v/v, 3.7% v/v, 3.75% v/v, 3.8% v/V, 3.85% v/v, 3.9% v/v, 3.95% v/v, 4% v/v, 4.05% v/v, 4.1% v/v, 4.15% v/v, 4.2% v/V, 4.25% v/v, 4.3% v/v, 4.35% v/v, 4.4% v/v, 4.45% v/v, 4.5% v/v, 4.55% v/v, 4.6% v/v, 4.65% v/v, 4.7% v/v, 4.75% v/v, 4.8% v/v, 4.85% v/v, 4.9% v/v, 4.95% v/v, or 5% v/v.

In some embodiments, the film forming agent is polyquaternium 11 (2-propenol acid, 2-methyl-2-(dimethlamino)ethyl ester, polymer and 1-ethenyl-2-pyrrolidinone, compound with diethyl sulfate; CAS #53633-54-8; also known as quaternium-23). In other embodiments, the film forming agent is another moisturizer safe for use on pet fur, safe for use on pet hair, and safe when it contacts pet skin.

### Emollients

In some embodiments, the curl cream or spray formulation includes one or more emollients, for example to increase emulsion stability and/or to reduce “crunchiness” in the pet's curly coat. When included, the emollients may be present in a total amount of 0.15% v/v to 25% v/v, for example 0.15% v/v, 0.2% v/v, 0.25% v/v, 0.3% v/v, 0.35% v/V, 0.4% v/v, 0.45% v/v, 0.5% v/v, 0.55% v/v, 0.6% v/v, 0.65% v/v, 0.7% v/v, 0.75% v/v, 0.8% v/v, 0.85% v/v, 0.9% v/v, 0.95% v/v, 1% v/v, 1.25% v/v, 1.5% v/v, 1.75% v/v, 2% v/v, 2.25% v/v, 2.5% v/v, 2.75% v/v, 3% v/v, 3.25% v/v, 3.5% v/v, 3.75% v/v, 4% v/V, 4.25% v/v, 4.5% v/v, 4.75% v/v, 5% v/v, 5.25% v/v, 5.5% v/v, 5.75% v/v, 6% v/v, 6.25% v/v, 6.5% v/v, 6.75% v/v, 7% v/v, 7.25% v/v, 7.5% v/v, 7.75% v/v, 8% v/v, 8.25% v/V, 8.5% v/v, 8.75% v/v, 9% v/v, 9.25% v/v, 9.5% v/v, 9.75% v/v, 10% v/v, 10.25% v/v, 10.5% v/v, 10.75% v/v, 11% v/v, 11.25% v/v, 11.5% v/v, 11.75% v/v, 12% v/v, 12.25% v/v, 12.5% v/v, 12.75% v/v, 13% v/v, 13.25% v/v, 13.5% v/v, 13.75% v/v, 14% v/V, 14.25% v/v, 14.5% v/v, 14.75% v/v, 15% v/v, 15.25% v/v, 15.5% v/v, 15.75% v/v, 16% v/v, 16.25% v/v, 16.5% v/v, 16.75% v/v, 17% v/v, 17.25% v/v, 17.5% v/v, 17.75% v/V, 18% v/v, 18.25% v/v, 18.5% v/v, 18.75% v/v, 19% v/v, 19.25% v/v, 19.5% v/v, 19.75% v/v, 20% v/v, 20.25% v/v, 20.5% v/v, 20.75% v/v, 21% v/v, 21.25% v/v, 21.5% v/V, 21.75% v/v, 22% v/v, 22.25% v/v, 22.5% v/v, 22.75% v/v, 23% v/v, 23.25% v/v, 23.5% v/v, 23.75% v/v, 24% v/v, 24.25% v/v, 24.5% v/v, 24.75% v/v, or 25% v/v.

In some embodiments, the curly cream formulation includes a single emollient. In other embodiments, the curly cream formulation includes two emollients. In other embodiments, the formulation includes three emollients. In other embodiments, the formulation includes four emollients. In still other embodiments, the formulation includes more than four emollients.

In some embodiments the curly cream formulation includes at least one emollient selected from the group consisting of: coco-caprylate/caprate, jojoba (Simmondsia chinensis) seed oil, panthenol (also known as pantothenol; 2,4-dihydroxy-N-(3-hydroxypropyl)-3,3-dimethylbutanamide), and caprylic/capric triglyceride. In some embodiments, the curly cream formulation includes only coco-caprylate/caprate and jojoba (Simmondsia chinensis) seed oil as emollients. In other embodiments, the curly cream formulation includes only jojoba (Simmondsia chinensis) seed oil, panthenol, and caprylic/capric triglyceride as emollients.

When present, the coco-caprylate/caprate emollient may be present in an amount of 0.5% v/v to 8% v/v, for example 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, 2% v/v, 2.1% v/v, 2.2% v/v, 2.3% v/v, 2.4% v/v, 2.5% v/v, 2.6% v/v, 2.7% v/v, 2.8% v/v, 2.9% v/v, 3% v/v, 3.1% v/v, 3.2% v/v, 3.3% v/v, 3.4% v/v, 3.5% v/v, 3.6% v/v, 3.7% v/v, 3.8% v/v, 3.9% v/v, 4% v/v, 4.1% v/v, 4.2% v/v, 4.3% v/v, 4.4% v/v, 4.5% v/v, 4.6% v/v, 4.7% v/v, 4.8% v/v, 4.9% v/v, 5% v/v, 5.1% v/v, 5.2% v/v, 5.3% v/v, 5.4% v/v, 5.5% v/v, 5.6% v/v, 5.7% v/v, 5.8% v/v, 5.9% v/v, 6% v/v, 6.1% v/v, 6.2% v/v, 6.3% v/v, 6.4% v/v, 6.5% v/v, 6.6% v/v, 6.7% v/v, 6.8% v/v, 6.9% v/v, 7% v/v, 7.1% v/v, 7.2% v/v, 7.3% v/v, 7.4% v/v, 7.5% v/v, 7.6% v/v, 7.7% v/v, 7.8% v/v, 7.9% v/v, or 8% v/v.

When present, the jojoba seed oil may be present in an amount of 0.1% v/v to 8% v/v, for example 0.1% v/v, 0.15% v/v, 0.2% v/v, 0.25% v/v, 0.3% v/v, 0.35% v/V, 0.4% v/v, 0.45% v/v, 0.5% v/v, 0.6% v/v, 0.7% v/v, 0.8% v/v, 0.9% v/v, 1% v/v, 1.1% v/V, 1.2% v/v, 1.3% v/v, 1.4% v/v, 1.5% v/v, 1.6% v/v, 1.7% v/v, 1.8% v/v, 1.9% v/V, 2% v/V, 2.1% v/v, 2.2% v/v, 2.3% v/v, 2.4% v/v, 2.5% v/v, 2.6% v/v, 2.7% v/V, 2.8% v/v, 2.9% v/V, 3% v/v, 3.1% v/v, 3.2% v/v, 3.3% v/v, 3.4% v/v, 3.5% v/v, 3.6% v/v, 3.7% v/v, 3.8% v/V, 3.9% v/v, 4% v/v, 4.1% v/v, 4.2% v/v, 4.3% v/v, 4.4% v/v, 4.5% v/v, 4.6% v/v, 4.7% v/V, 4.8% v/v, 4.9% v/v, 5% v/v, 5.1% v/v, 5.2% v/v, 5.3% v/v, 5.4% v/v, 5.5% v/v, 5.6% v/V, 5.7% v/v, 5.8% v/v, 5.9% v/v, 6% v/v, 6.1% v/v, 6.2% v/v, 6.3% v/v, 6.4% v/v, 6.5% v/V, 6.6% v/v, 6.7% v/v, 6.8% v/v, 6.9% v/v, 7% v/v, 7.1% v/v, 7.2% v/v, 7.3% v/v, 7.4% v/V, 7.5% v/v, 7.6% v/v, 7.7% v/v, 7.8% v/v, 7.9% v/v, or 8% v/v.

When present, the panthenol may be present in an amount of 0.01% v/v to 5% v/v, for example 0.01% v/v, 0.02% v/v, 0.03% v/v, 0.04% v/v, 0.05% v/v, 0.06% v/V, 0.07% v/v, 0.08% v/v, 0.09% v/v, 0.1% v/v, 0.15% v/v, 0.2% v/v, 0.25% v/v, 0.3% v/V, 0.35% v/v, 0.4% v/v, 0.45% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, 2% v/v, 2.1% v/v, 2.2% v/v, 2.3% v/v, 2.4% v/v, 2.5% v/v, 2.6% v/v, 2.7% v/v, 2.8% v/v, 2.9% v/v, 3% v/v, 3.1% v/v, 3.2% v/v, 3.3% v/v, 3.4% v/v, 3.5% v/v, 3.6% v/v, 3.7% v/v, 3.8% v/v, 3.9% v/v, 4% v/v, 4.1% v/v, 4.2% v/v, 4.3% v/v, 4.4% v/v, 4.5% v/v, 4.6% v/v, 4.7% v/v, 4.8% v/v, 4.9% v/v, or 5% v/v.

When present, the caprylic/capric triglyceride emollient may be present in an amount of 0.1% v/v to 15% v/v, for example 0.1% v/v, 0.2% v/v, 0.3% v/v, 0.33% v/V, 0.4% v/v, 0.5% v/v, 0.6% v/v, 0.7% v/v, 0.8% v/v, 0.9% v/v, 1% v/v, 1.1% v/v, 1.2% v/V, 1.3% v/v, 1.4% v/v, 1.5% v/v, 1.6% v/v, 1.7% v/v, 1.8% v/v, 1.9% v/v, 2% v/v, 2.1% v/V, 2.2% v/v, 2.3% v/v, 2.4% v/v, 2.5% v/v, 2.6% v/v, 2.7% v/v, 2.8% v/v, 2.9% v/v, 3% v/V, 3.1% v/v, 3.2% v/v, 3.3% v/v, 3.4% v/v, 3.5% v/v, 3.6% v/v, 3.7% v/v, 3.8% v/v, 3.9% v/V, 4% v/v, 4.1% v/v, 4.2% v/v, 4.3% v/v, 4.4% v/v, 4.5% v/v, 4.6% v/v, 4.7% v/v, 4.8% v/V, 4.9% v/v, 5% v/v, 5.1% v/v, 5.2% v/v, 5.3% v/v, 5.4% v/v, 5.5% v/v, 5.6% v/v, 5.7% v/V, 5.8% v/v, 5.9% v/v, 6% v/v, 6.1% v/v, 6.2% v/v, 6.3% v/v, 6.4% v/v, 6.5% v/v, 6.6% v/V, 6.7% v/v, 6.8% v/v, 6.9% v/v, 7% v/v, 7.1% v/v, 7.2% v/v, 7.3% v/v, 7.4% v/v, 7.5% v/V, 7.6% v/v, 7.7% v/v, 7.8% v/v, 7.9% v/v, 8% v/v, 8.1% v/v, 8.2% v/v, 8.3% v/v, 8.4% v/V, 8.5% v/v, 8.6% v/v, 8.7% v/v, 8.8% v/v, 8.9% v/v, 9% v/v, 9.1% v/v, 9.2% v/v, 9.3% v/V, 9.4% v/v, 9.5% v/v, 9.6% v/v, 9.7% v/v, 9.8% v/v, 9.9% v/v, 10% v/v, 10.1% v/v, 10.2% v/v, 10.3% v/v, 10.4% v/v, 10.5% v/v, 10.6% v/v, 10.7% v/v, 10.8% v/v, 10.9% v/v, 11% v/v, 11.1% v/v, 11.2% v/v, 11.3% v/v, 11.4% v/v, 11.5% v/v, 11.6% v/v, 11.7% v/V, 11.8% v/v, 11.9% v/v, 12% v/v, 12.1% v/v, 12.2% v/v, 12.3% v/v, 12.4% v/v, 12.5% v/V, 12.6% v/v, 12.7% v/v, 12.8% v/v, 12.9% v/v, 13% v/v, 13.1% v/v, 13.2% v/v, 13.3% v/V, 13.4% v/v, 13.5% v/v, 13.6% v/v, 13.7% v/v, 13.8% v/v, 13.9% v/v, 14% v/v, 14.1% v/V, 14.2% v/v, 14.3% v/v, 14.4% v/v, 14.5% v/v, 14.6% v/v, 14.7% v/v, 14.8% v/v, 14.9% v/v, or 15% v/V.

In embodiments that include both coco-caprylate/caprate and jojoba seed oil as emollients, the ratio of coco-caprylate/caprate to jojoba seed oil may be 1:16 to 80:1, for example 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, or 80:1.

In embodiments that include panthenol as an emollient, the ratio of panthenol to coco-caprylate/caprate may be 1:80 to 10:1, for example 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In embodiments that include caprylic/capric triglyceride and coco-caprylate/caprate as an emollients, the ratio of caprylic/capric triglyceride to coco-caprylate/caprate may be 1:16 to 30:1, for example 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1.

In embodiments that include panthenol and coco-caprylate/caprate as emollients, the ratio of panthenol to coco-caprylate/caprate may be 1:80 to 10:1, for example 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

### Preservatives

In some embodiments, the curl cream or spray formulation includes one or more preservatives, for example to increase shelf life of the formulation. When included, the preservatives may be present in a total amount of 0.05% v/v to 4% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.21% v/v, 0.22% v/v, 0.23% v/v, 0.24% v/v, 0.25% v/v, 0.26% v/v, 0.27% v/v, 0.28% v/v, 0.29% v/v, 0.3% v/v, 0.31% v/v, 0.32% v/v, 0.33% v/v, 0.34% v/v, 0.35% v/v, 0.36% v/v, 0.37% v/v, 0.38% v/v, 0.39% v/v, 0.4% v/v, 0.41% v/v, 0.42% v/v, 0.43% v/v, 0.44% v/v, 0.45% v/v, 0.46% v/v, 0.47% v/v, 0.48% v/v, 0.49% v/v, 0.5% v/v, 0.51% v/v, 0.52% v/v, 0.53% v/v, 0.54% v/v, 0.55% v/v, 0.56% v/v, 0.57% v/v, 0.58% v/v, 0.59% v/v, 0.6% v/v, 0.61% v/v, 0.62% v/v, 0.63% v/v, 0.64% v/v, 0.65% v/v, 0.66% v/v, 0.67% v/V, 0.68% v/v, 0.69% v/v, 0.7% v/v, 0.71% v/v, 0.72% v/v, 0.73% v/v, 0.74% v/v, 0.75% v/v, 0.76% v/v, 0.77% v/v, 0.78% v/v, 0.79% v/v, 0.8% v/v, 0.81% v/v, 0.82% v/v, 0.83% v/V, 0.84% v/v, 0.85% v/v, 0.86% v/v, 0.87% v/v, 0.88% v/v, 0.89% v/v, 0.9% v/v, 0.91% v/v, 0.92% v/v, 0.93% v/v, 0.94% v/v, 0.95% v/v, 0.96% v/v, 0.97% v/v, 0.98% v/v, 0.99% v/v, 1% v/v, 1% v/v, 1.25% v/v, 1.5% v/v, 1.75% v/v, 2% v/v, 2.25% v/v, 2.5% v/v, 2.75% v/v, 3% v/v, 3.25% v/v, 3.5% v/v, 3.75% v/v, or 4% v/V.

In some embodiments, the curly cream formulation includes a single preservative. In other embodiments, the curly cream formulation includes two preservatives. In other embodiments, the formulation includes three preservatives. In still other embodiments, the formulation includes four preservatives. In some embodiments, the formulation includes more than four preservatives.

In some embodiments the curly cream formulation includes at least one preservative selected from the group consisting of: phenoxyethanol, ethylhexylglycerin, dehydroacetic acid, benzyl alcohol, potassium sorbate, Hexacon 91 (phenoxyethanol/ethylhexylglycerin), and Geoguard 221 (dehydroacetic acid/benzyl alcohol).

In some embodiments, the curly cream formulation includes (e.g., only includes) phenoxyethanol and ethylhexylglycerin as preservatives. In some embodiments, the curly cream formulation includes (e.g., only includes) dehydroacetic acid and benzyl alcohol as preservatives. In some embodiments, the curly cream formulation includes (e.g., only includes) phenoxyethanol, ethylhexylglycerin, and potassium sorbate as preservatives. In some embodiments, the curly cream formulation includes (e.g., only includes) dehydroacetic acid, benzyl alcohol, and potassium sorbate as preservatives. In some embodiments, the curly cream formulation includes (e.g., only includes) Hexacon 91 as a preservative. In some embodiments, the curly cream formulation includes (e.g., only includes) Geoguard 221 as a preservative. In some embodiments, the curly cream formulation includes (e.g., only includes) Geoguard 221 and potassium sorbate as preservatives.

When included, the phenoxyethanol preservative may be present in an amount of 0.2% v/v to 2% v/v, for example 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/V.

When included, the ethylhexylglycerin preservative may be present in an amount of 0.2% v/v to 2% v/v, for example 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

When included, the dehydroacetic acid preservative may be present in an amount of 0.05% v/v to 2% v/v, for example 0.05% v/v, 0.06% v/v, 0.07% v/v, 0.08% v/V, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

When included, the benzyl alcohol preservative may be present in an amount of 0.05% v/v to 2% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/V, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v. In some embodiments, the benzyl alcohol preservative is present in an amount not more than 1% v/v, such as 1% v/v, 0.9% v/v, 0.8% v/v, 0.7% v/v, 0.6% v/v, 0.5% v/v, 0.4% v/v, 0.3% v/v, 0.2% v/v, 0.1% v/v, 0.05% v/v, or less than 0.05% v/v.

When included, the potassium sorbate preservative may be present in an amount of 0.05% v/v to 1% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/V, 0.8% v/v, 0.9% v/v, or 1% v/V.

When included, the Hexacon 91 preservative may be present in an amount of 0.2% v/v to 2% v/v, for example 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

When included, the Geoguard 221 preservative may be present in an amount of 0.05% v/v to 2% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/V, 0.8% v/v, 0.9% v/v, 1% v/v, 1.1% v/v, 1.2% v/v, 1.3% v/v, 1.4% v/v, 1.5% v/v, 1.6% v/V, 1.7% v/v, 1.8% v/v, 1.9% v/v, or 2% v/v. In some embodiments, the Geoguard 221 preservative is present in an amount sufficient to provide benzyl alcohol in an amount of 0.05% v/v to 2% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/V, 0.8% v/v, 0.9% v/v, 1% v/v, 1.1% v/v, 1.2% v/v, 1.3% v/v, 1.4% v/v, 1.5% v/v, 1.6% v/V, 1.7% v/v, 1.8% v/v, 1.9% v/v, or 2% v/v. In some embodiments, the Geoguard 221 preservative is present in an amount sufficient to provide benzyl alcohol in an amount not more than 1% v/v, such as 1% v/v, 0.9% v/v, 0.8% v/v, 0.7% v/v, 0.6% v/v, 0.5% v/V, 0.4% v/v, 0.3% v/v, 0.2% v/v, 0.1% v/v, 0.05% v/v, or less than 0.05% v/v.

### Antimicrobial Agents

In some embodiments, the curly cream formulation includes at least one antimicrobial agent. When included, the antimicrobial agent(s) may be present in a total amount of 0.05% v/v to 5% v/v, for example 0.05% v/v, 0.06% v/v, 0.07% v/v, 0.08% v/V, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, 2% v/v, 2.1% v/v, 2.2% v/v, 2.3% v/v, 2.4% v/v, 2.5% v/v, 2.6% v/v, 2.7% v/v, 2.8% v/v, 2.9% v/v, 3% v/v, 3.1% v/v, 3.2% v/v, 3.3% v/v, 3.4% v/v, 3.5% v/v, 3.6% v/v, 3.7% v/v, 3.8% v/v, 3.9% v/v, 4% v/v, 4.1% v/v, 4.2% v/v, 4.3% v/v, 4.4% v/v, 4.5% v/v, 4.6% v/v, 4.7% v/v, 4.8% v/v, 4.9% v/v, or 5% v/V.

In some embodiments, the curly cream formulation includes a single antimicrobial agent. In other embodiments, the curly cream formulation includes two antimicrobial agents. In other embodiments, the formulation includes three antimicrobial agents. In still other embodiments, the formulation includes four antimicrobial agents. In some embodiments, the formulation includes more than four antimicrobial agents.

In some embodiments the curly cream formulation includes at least one antimicrobial agent selected from the group consisting of: neem (Azadirachta indica) seed oil, and Citrus grandis seed extract. In some embodiments, the curly cream formulation only includes neem seed oil as the antimicrobial agent. In other embodiments, the curly cream formulation only includes Citrus grandis seed extract as the antimicrobial agent.

When included, the need seed oil antimicrobial agent may be present in an amount of 0.05% v/v to 4% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, 2% v/v, 2.1% v/v, 2.2% v/v, 2.3% v/v, 2.4% v/v, 2.5% v/v, 2.6% v/v, 2.7% v/v, 2.8% v/v, 2.9% v/v, 3% v/v, 3.1% v/v, 3.2% v/v, 3.3% v/v, 3.4% v/v, 3.5% v/v, 3.6% v/v, 3.7% v/v, 3.8% v/v, 3.9% v/v, or 4% v/v.

When included, the Citrus grandis seed extract antimicrobial agent may be present in an amount of 0.05% v/v to 4% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/V, 0.6% v/v, 0.7% v/v, 0.8% v/v, 0.9% v/v, 1% v/v, 1.1% v/v, 1.2% v/v, 1.3% v/v, 1.4% v/V, 1.5% v/v, 1.6% v/v, 1.7% v/v, 1.8% v/v, 1.9% v/v, 2% v/v, 2.1% v/v, 2.2% v/v, 2.3% v/V, 2.4% v/v, 2.5% v/v, 2.6% v/v, 2.7% v/v, 2.8% v/v, 2.9% v/v, 3% v/v, 3.1% v/v, 3.2% v/V, 3.3% v/v, 3.4% v/v, 3.5% v/v, 3.6% v/v, 3.7% v/v, 3.8% v/v, 3.9% v/v, or 4% v/v.

### Perfumes

In some embodiments, the curly cream or spray formulation includes at least one perfume (perfuming agent). When included, the perfume(s) may be present in a total amount of 0.01% v/v to 2% v/v, for example 0.01% v/v, 0.02% v/v, 0.03% v/v, 0.04% v/V, 0.05% v/v, 0.06% v/v, 0.07% v/v, 0.08% v/v, 0.09% v/v, 0.1% v/v, 0.11% v/v, 0.12% v/V, 0.13% v/v, 0.14% v/v, 0.15% v/v, 0.16% v/v, 0.17% v/v, 0.18% v/v, 0.19% v/v, 0.2% v/V, 0.3% v/v, 0.4% v/v, 0.5% v/v, 0.6% v/v, 0.7% v/v, 0.8% v/v, 0.9% v/v, 1% v/v, 1.1% v/V, 1.2% v/v, 1.3% v/v, 1.4% v/v, 1.5% v/v, 1.6% v/v, 1.7% v/v, 1.8% v/v, 1.9% v/v, or 2% v/v.

Any suitable perfume may be included in a curly cream or spray formulation. In some embodiments, the perfume is Anthemis nobilis flower oil. In other embodiments, the perfume is Lavandula angustifolia oil. In other embodiments, the perfume is a combination of Anthemis nobilis flower oil and Lavandula angustifolia oil.

The inclusion of a perfume in curl cream and curl spray formulations consistent with the present disclosure is optional. In some embodiments, for example, a curl cream or curl spray formulation includes no perfume (perfuming agent).

### Gelling Agents

In some embodiments, the curly cream formulation includes at least one gelling agent, for example to increase the strength and duration that the curly cream adheres to the pet fur/hair. When included, the gelling agent(s) may be present in a total amount of 0.05% v/v to 2% v/v, for example 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/v, 0.7% v/V, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

In some embodiments, the gelling agent is selected from the group consisting of: polyacrylic acid, carboxymethylcellulose sodium, hydroxyethyl cellulose, hydroxypropyl cellulose, methylcellulose, hydroxypropyl methylcellulose, and Poloxamer 407. In some embodiments, the gelling agent is a polyacrylic acid (Carbomer), such as Carbomer 940 or Carbomer 934. In other embodiments, the gelling agent is carboxymethylcellulose sodium. In some embodiments, the gelling agent is hydroxyethyl cellulose. In some embodiments, the gelling agent is hydroxypropyl cellulose. In some embodiments, the gelling agent is methylcellulose. In some embodiments, the gelling agent is hydroxypropyl methylcellulose. In some embodiments, the gelling agent is Poloxamer 407.

### Chelating Agents

In some embodiments, the curly cream formulation includes at least one chelating agent, for example to increase stability of the formulation. When included, the chelating agent(s) may be present in a total amount of 0.01% v/v to 1% v/v, for example 0.01% v/v, 0.02% v/v, 0.03% v/v, 0.04% v/v, 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/v, 0.6% v/V, 0.7% v/v, 0.8% v/v, 0.9% v/v, or 1% v/V.

In some embodiments, the chelating agent is selected from the group consisting of: disodium EDTA, Barbary fig stem extract, caprylhydroxamic acid, citric acid, Crataegus oxyacantha (Hawthorn) flower extract, gluconolactone, kojic acid, lactobionic acid, Moringa oleifera (moringaceae) seed extract, phytic acid, sodium citrate, sorrel (Rumex acetosa) leaf extract, tetrahydroxypropyl ethylenediamine, and tetrasodium EDTA. In some embodiments, the gelling agent is disodium EDTA. In some embodiments, the chelating agent is Barbary fig stem extract. In some embodiments, the chelating agent is caprylhydroxamic acid. In some embodiments, the chelating agent is citric acid. In some embodiments, the chelating agent is Crataegus oxyacantha (Hawthorn) flower extract. In some embodiments, the chelating agent is gluconolactone. In some embodiments, the chelating agent is kojic acid. In some embodiments, the chelating agent is lactobionic acid. In some embodiments, the chelating agent is Moringa oleifera (moringaceae) seed extract. In some embodiments, the chelating agent is phytic acid. In some embodiments, the chelating agent is sodium citrate. In some embodiments, the chelating agent is sorrel (Rumex acetosa) leaf extract. In some embodiments, the chelating agent is tetrahydroxypropyl ethylenediamine. In some embodiments, the chelating agent is and tetrasodium EDTA.

### Antistatic Agents

In some embodiments, the curly cream formulation includes at least one antistatic agent, for example to enhance the detangling effects of the formulation. When included, the antistatic agent(s) may be present in a total amount of 0.01% v/v to 1% v/v, for example 0.01% v/v, 0.02% v/v, 0.03% v/v, 0.04% v/v, 0.05% v/v, 0.06% v/v, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/v, 0.5% v/V, 0.6% v/v, 0.7% v/v, 0.8% v/v, 0.9% v/v, or 1% v/v.

In some embodiments, the antistatic agent is cetrimonium chloride.

### Antioxidants

In some embodiments, the curly cream formulation includes at least one antioxidant, for example to attenuate the decomposition of the formulation from oxidants. When included, the antioxidant(s) may be present in a total amount of 0.01% v/v to 2% v/v, for example 0.01% v/v, 0.02% v/v, 0.03% v/v, 0.04% v/v, 0.05% v/v, 0.06% v/V, 0.07% v/v, 0.08% v/v, 0.09% v/v, 0.1% v/v, 0.11% v/v, 0.12% v/v, 0.13% v/v, 0.14% v/v, 0.15% v/v, 0.16% v/v, 0.17% v/v, 0.18% v/v, 0.19% v/v, 0.2% v/v, 0.3% v/v, 0.4% v/V, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

In some embodiments, the antioxidant is tocopherol (e.g., α-tocopherol).

### pH Adjusters & Buffering Agents

In some embodiments, the curly cream formulation includes at least one pH adjuster, for example to adjust the pH level of the formulation to a desired initial pH level. When included, the pH adjuster is included in an amount sufficient to adjust the pH of the formulation to an initial pH value, such as a pH level within the range of 4.4 to 8.1, preferably within the range of 6.0 to 7.5, or more preferably to an initial pH level of about 6.5.

In some embodiments, the pH adjuster is a weak base. In some embodiments, the pH adjuster is triethanolamine.

In some embodiments, the curly cream formulation includes at least one buffering agent, for example to improve the formulations resistance to pH drift over time. When included, the buffering agent is included in an amount sufficient to maintain the pH of the formulation at or near the formulation's initial pH value, for example an initial pH level within the range of 4.4 to 8.1, preferably within the range of 6.0 to 7.5, or more preferably about 6.0 or 6.5.

In some embodiments, the buffering agent is citric acid.

In embodiments that include a buffering agent, the curly cream formulation may be stable upon storage for at least 6 months or for at least 12 months. For example and without limitation, the curly cream formulation, after storage at 25 °C and 60% relative humidity for at least 12 months, may have a pH level that is within 1.0 pH unit of its initial pH level (i.e., when first prepared). In other embodiments, the curly cream formulation, after storage at 25 °C and 60% relative humidity for at least 12 months, may have a pH level that is within 0.5 pH unit of its initial pH level (i.e., when first prepared). In some embodiments, the curly cream formulation, after storage at 40 °C and 75% relative humidity for at least 6 months, may have a pH level after storage for at least 12 months that is within 1.0 pH unit of its initial pH level (i.e., when first prepared). In other embodiments, the curly cream formulation, after storage at 40 °C and 75% relative humidity for at least 6 months, may have a pH level after storage that is within 0.5 pH unit of its initial pH level (i.e., when first prepared).

### Solubilizers

In some embodiments, the curl cream or spray formulation includes one or more solubilizers, for example to improve solubility of one or more other components of the formulation in the solvent. When included, the solubilizer(s) may be present in a total amount of 0.5% v/v to 10% v/v, for example 0.5% v/v, 0.6% v/v, 0.7% v/v, 0.8% v/V, 0.9% v/v, 1% v/v, 1.1% v/v, 1.2% v/v, 1.3% v/v, 1.4% v/v, 1.5% v/v, 1.6% v/v, 1.7% v/V, 1.8% v/v, 1.9% v/v, 2% v/v, 2.1% v/v, 2.2% v/v, 2.3% v/v, 2.4% v/v, 2.5% v/v, 2.6% v/V, 2.7% v/v, 2.8% v/v, 2.9% v/v, 3% v/v, 3.1% v/v, 3.2% v/v, 3.3% v/v, 3.4% v/v, 3.5% v/V, 3.6% v/v, 3.7% v/v, 3.8% v/v, 3.9% v/v, 4% v/v, 4.1% v/v, 4.2% v/v, 4.3% v/v, 4.4% v/V, 4.5% v/v, 4.6% v/v, 4.7% v/v, 4.8% v/v, 4.9% v/v, or 5% v/V.

In some embodiments, the curly cream formulation includes a single solubilizer. In other embodiments, the curly cream formulation includes two solubilizers. In other embodiments, the formulation includes three solubilizers. In still other embodiments, the formulation includes four solubilizers. In some embodiments, the formulation includes more than four solubilizers.

In some embodiments the curly cream formulation includes at least one solubilizer selected from the group consisting of: beta-glucan, pectin, and BergaMuls ET (beta-glucan/pectin).

In some embodiments, the curly cream formulation includes (e.g., only includes) beta-glucan as a solubilizer. In some embodiments, the curly cream formulation includes (e.g., only includes) pectin as a solubilizer. In some embodiments, the curly cream formulation includes (e.g., only includes) beta-glucan and pectin as solubilizers. In some embodiments, the curly cream formulation includes (e.g., only includes) BergaMuls ET1 as the solubilizer.

### Viscosity Controlling Agents

In some embodiments, the curly cream formulation includes at least one viscosity controlling agent, for example to modify or stabilize the viscosity of the formulation. When included, the viscosity controlling agent(s) may be present in a total amount of 0.1% v/v to 10% v/v, for example 0.1% v/v, 0.15% v/v, 0.2% v/v, 0.25% v/v, 0.3% v/V, 0.35% v/v, 0.4% v/v, 0.45% v/v, 0.5% v/v, 0.55% v/v, 0.6% v/v, 0.65% v/v, 0.7% v/v, 0.75% v/v, 0.8% v/v, 0.85% v/v, 0.9% v/v, 0.95% v/v, 1% v/v, 1.25% v/v, 1.5% v/v, 1.75% v/v, 2% v/v, 2.25% v/v, 2.5% v/v, 2.75% v/v, 3% v/v, 3.25% v/v, 3.5% v/v, 3.75% v/v, 4% v/v, 4.25% v/v, 4.5% v/v, 4.75% v/v, 5% v/v, 5.25% v/v, 5.5% v/v, 5.75% v/v, 6% v/v, 6.25% v/v, 6.5% v/v, 6.75% v/v, 7% v/v, 7.25% v/v, 7.5% v/v, 7.75% v/v, 8% v/V, 8.25% v/v, 8.5% v/v, 8.75% v/v, 9% v/v, 9.25% v/v, 9.5% v/v, 9.75% v/v, or 10% v/v.

In some embodiments, the viscosity controlling agent is cetyl alcohol.

### Solvents

In some embodiments, the curl cream or spray formulation includes water as a solvent. In other embodiments, the curl cream or spray formulation includes glycerin as a solvent. In some embodiments, the solvent is a solvent system including more than one solvent, for example a combination of water and glycerin.

In some embodiments, glycerin is present as a solvent in a curl cream or spray formulation consistent with the present disclosure through the inclusion of another type of component. For example and without limitation, an embodiment consistent with the present disclosure that includes linseed extract may also necessarily include a small amount of glycerin if the linseed extract used to prepare the curl cream or spray formulation itself includes glycerin. Similarly, an embodiment consistent with the present disclosure that includes marshmallow root extract may also necessarily include a small amount of glycerin if the marshmallow root extract used to prepare the curl cream or spray formulation itself includes glycerin.

### Methods of Making Curl Cream and Curl Spray Formulations

In embodiments that include arginine as a conditioner and carbomer as a gelling agent, a curl cream or spray formulation is prepared by combining the powdered arginine and carbomer components, along with the chelating agent (if present) and the antistatic agent (if present) together before adding them to water and heating the mixture to at least 60 °C but not more than 80 °C with stirring until homogenized. The homogenized mixture is then cooled to 60 °C whereupon the linseed extract, marshmallow root extract, aloe barbadensis leaf juice and slippery elm extract (if present) is added to the homogenized mixture. Separately, the emollients and antimicrobial agent (if present) are combined and heated to 60-80 °C before cooling to below 60 °C. The two mixtures are then combined and thoroughly mixed before allowing the combined mixture to cool to 40-45 °C. All remaining components are then added to the combined mixture and blended until homogenized.

In embodiments that include silk amino acids as a conditioner and carbomer as a gelling agent, the curl cream or spray formulation is prepared by adding the chelating agent (if present), and the antistatic agent (if present), one-by-one to stirring water (e.g., at about 400 rpm), ensuring that each component is fully dissolved before adding the next component. At increased stirring rate (e.g., 800-1,200 rpm), the powdered carbomer is added slowly by sifting, followed by additional vigorous agitation. The pH is then adjusted to 6.5 with dropwise addition of triethanolamine. The mixture should now resemble a clear gel. Separately, the beta-glucan/pectin (e.g., BergaMuls ET1) is added to the linseed extract, slippery elm extract, aloe barbadensis leaf juice, marshmallow root extract, the film-forming agent (e.g., polyquaternium 11), and the preservative (e.g., Geoguard 21; dehydroacetic acid/benzyl alcohol), and the mixture is stirred until fully dispersed. The two mixtures are then combined gradually while homogenizing until a uniform lotion is obtained. The silk amino acids (conditioner) are then added to the lotion and mixed until thoroughly combined (typically at least 15 minutes). Additional triethanolamine may be added to achieve an initial pH of 4.4 to 8.1, preferably 6.0 to 7.5, and more preferably 6.5.

In embodiments that include guar hydroxypropyltrimonium chloride as a conditioner, the curl cream or spray formulation is prepared by adding the antistatic agent (if present), glycerin humectant (if present), and panthenol emollient (if present) to stirring water (e.g., at 400 rpm) until all solids are dissolved. The mixture is then heated to 70-75 °C. Separately, jojoba seed oil emollient, caprylic/capric triglyceride emollient, the viscosity controlling agent(s) and the emulsifier(s) are combined and heated to 70-75 °C until fully melted. Both mixtures are maintained separately at 70-75 °C for at least 10 minutes. While the first mixture is stirred at about 600 rpm at 70-75 °C, the guar hydroxypropyltrimonium chloride is added and stirred for an additional 10 minutes at 70-75 °C before the heated second mixture is added. After at least 15 minutes of stirring at 70-75 °C, the combined mixture is allowed to cool to under 45 °C while being stirred. When the mixture reaches a temperature of 40-45 °C, the linseed extract conditioning agent and the slippery elm moisturizing agent are added and stirred until uniformly mixed. When the mixture then reaches 38-40 °C, the humectant, the remaining conditioning agent(s), the film forming agent, the preservative, and the perfume are added and mixed until uniform. The mixture is then stirred for an additional 15 minutes to ensure complete distribution of all components. If needed, the pH can be adjusted using citric acid (50% in water) to achieve an initial pH of 4.4 to 8.1, preferably 6.0 to 7.5, and more preferably 6.5.

Several example curl cream and curl spray formulations consistent with the present disclosure and prepared by the methods described above are set forth below.

TABLE 1
Formula V1

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Humectant | Aloe barbadensis leaf | 0.1-50% |
| | juice | |
| Emollient 1 | Coco-caprylate/ | 0.5-5% |
| | caprate | |
| Emollient 2 | Jojoba seed oil | 0.1-5% |
| Preservative 1 | Geoguard 221 | 0.2-1% |
| | (decahydroacetic acid, | |
| | benzyl alcohol) | |
| Preservative 2 | Potassium sorbate | 0.1-0.5% |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Antioxidant | Tocopherol | 0.05-1% |
| Antimicrobial | Citrus grandis seed | 0.1-2% |
| agent | extract | |
| Solvent | Water | 44-90% |

TABLE 2
Formula V1.2

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Moisturizer | Slippery elm extract | 0.5-10% |
| Humectant | Aloe barbadensis leaf | 0.1-50% |
| | juice | |
| Emollient 1 | Coco-caprylate/ | 0.5-5% |
| | caprate | |
| Emollient 2 | Jojoba seed oil | 0.1-5% |
| Preservative 1 | Geoguard 221 | 0.2-1.15% |
| | (decahydroacetic acid, | |
| | benzyl alcohol) | |
| Preservative 2 | Potassium sorbate | 0.1-0.5% |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Antioxidant | Tocopherol | 0.05-1% |
| Antimicrobial | Citrus grandis seed | 0.1-2% |
| agent | extract | |
| Solvent | Water | 44-90% |

TABLE 3
Formula V1.3

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Moisturizer | Slippery elm extract | 0.5-10% |
| Humectant | Aloe barbadensis leaf | 0.1-50% |
| | juice | |
| Emollient 1 | Coco-caprylate/ | 0.5-5% |
| | caprate | |
| Emollient 2 | Jojoba seed oil | 0.1-5% |
| Preservative | Hexacon 91 | 0.5-1.1% |
| | (phenoxyethanol, | |
| | ethylhexylglycerin) | |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Antimicrobial | Neem seed oil | 0-2% |
| agent | | |
| Solvent | Water | 44-90% |

TABLE 4
Formula V2

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Moisturizer | Slippery elm extract | 0.5-10% |
| Humectant | Aloe barbadensis leaf | 0.1-50% |
| | juice | |
| Film forming | Polyquaternium 11 | 0.2-3% |
| agent | | |
| Emollient 1 | Coco-caprylate/ | 0.5-5% |
| | caprate | |
| Emollient 2 | Jojoba seed oil | 0.1-5% |
| Preservative | Hexacon 91 | 0.5-1.1% |
| | (phenoxyethanol, | |
| | ethylhexylglycerin) | |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Antimicrobial | Neem seed oil | 0-2% |
| agent | | |
| Solvent | Water | 44-90% |

TABLE 5
Formula V2.2

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Moisturizer | Slippery elm extract | 0.5-10% |
| Humectant | Aloe barbadensis leaf | 0.1-50% |
| | juice | |
| Film forming | Polyquaternium 11 | 0.2-3% |
| agent | | |
| Emollient 1 | Coco-caprylate/ | 0.5-5% |
| | caprate | |
| Emollient 2 | Jojoba seed oil | 0.1-5% |
| Preservative | Geoguard 221 | 0.2-1% |
| | (decahydroacetic acid, | |
| | benzyl alcohol) | |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Antimicrobial | Neem seed oil | 0-2% |
| agent | | |
| Solvent | Water | 44-90% |

TABLE 6
Formula V3

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Moisturizer | Slippery elm extract | 0.5-10% |
| Humectant | Aloe barbadensis leaf | 0.1-50% |
| | juice | |
| Film forming | Polyquaternium 11 | 0.2-3% |
| agent | | |
| Emollient 1 | Coco-caprylate/ | 0.5-5% |
| | caprate | |
| Emollient 2 | Jojoba seed oil | 0.1-5% |
| Preservative | Geoguard 221 | 0.2-1% |
| | (decahydroacetic acid, | |
| | benzyl alcohol) | |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Chelating | Disodium EDTA | 0.02-0.5% |
| agent | | |
| Antistatic | Cetrimonium chloride | 0.11-0.833% |
| agent | | |
| Antimicrobial | Neem seed oil | 0-2% |
| agent | | |
| Solvent | Water | 44-90% |

TABLE 7
Formula V4

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Silk amino acids | 0.33-5% |
| Moisturizer | Slippery elm extract | 0.5-10% |
| Humectant | Aloe barbadensis leaf | 0.1-50% |
| | juice | |
| Film forming | Polyquaternium 11 | 0.2-3% |
| agent | | |
| Emollient 1 | Coco-caprylate/ | 0.5-5% |
| | caprate | |
| Emollient 2 | Jojoba seed oil | 0.1-5% |
| Preservative | Geoguard 221 | 0.2-1% |
| | (decahydroacetic acid, | |
| | benzyl alcohol) | |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Chelating | Disodium EDTA | 0.02-0.5% |
| agent | | |
| Antistatic | Cetrimonium chloride | 0.11-0.833% |
| agent | | |
| pH adjuster | Triethanolamine | q.s. |
| | | (about 0.1-1%) |
| Solubilizer | Beta-glucan, pectin | 0.50-5% |
| Solvent | Water | 44-90% |

TABLE 8
Formula V5

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Silk amino acids | .033-5% |
| Conditioner 4 | Guar hydroxypropyl- | 0.07-2% |
| | trimonium chloride | |
| Moisturizer | Slippery elm extract | 0.5-10% |
| Humectant 1 | Aloe barbadensis leaf | 0.1-50% |
| | juice | |
| Humectant 2 | Glycerin | 1.96-5.155% |
| Film forming | Polyquaternium 11 | 0.2-3% |
| agent | | |
| Emollient 1 | Jojoba seed oil | 0.1-5% |
| Emollient 2 | Panthenol | 0.07-5% |
| Emollient 3 | Caprylic/Capric | 0.33-10% |
| | triglyceride | |
| Preservative | Geoguard 221 | 0.2-1% |
| | (decahydroacetic acid, | |
| | benzyl alcohol) | |
| Perfume | Anthemis Nobilis Flower | 0-1% |
| | Oil/Parfum/ | |
| | Lavandula Angustifolia | |
| | Oil | |
| Emulsifier 1 | Cetearyl alcohol, | 0.33-5% |
| | behentrimonium | |
| | methosulfate | |
| Emulsifier 2 | Glyceryl stearate | 0.33-7% |
| Antistatic | Cetrimonium chloride | 0.11-0.833% |
| agent | | |
| Buffering | Citric acid | q.s. |
| agent | | (about 0.1-1%) |
| Solvent | Water | 44-90% |

TABLE 9
Formula V6

| Component | Species | Concentration |
|---|---|---|
| Conditioner 1 | Linseed extract | 1-20.5% |
| Conditioner 2 | Marshmallow root | 0.4-24% |
| | extract | |
| Conditioner 3 | Arginine (base) | 0.1-4% |
| Moisturizer | Slippery elm extract | 0.5-10% |
| Humectant | Aloe barbadensis leaf | 0.1-50% |
| | juice | |
| Film forming | Polyquaternium 11 | 0.2-3% |
| agent | | |
| Emollient 1 | Coco-caprylate/ | 0.5-5% |
| | caprate | |
| Emollient 2 | Jojoba seed oil | 0.1-5% |
| Emulsifier/ | Polysorbate 20 | 1-10% |
| Solubilizer | | |
| Preservative | Hexacon 91 | 0.5-1.1% |
| | (phenoxyethanol, | |
| | ethylhexylglycerin) | |
| Perfume | Anthemis Nobilis Flower | 0-1% |
| | Oil/Parfum/ | |
| | Lavandula Angustifolia | |
| | Oil | |
| Gelling agent | Carbomer 940 | 0.1-1% |
| Antimicrobial | Neem seed oil | 0-2% |
| agent | | |
| Solvent | Water | 34-90% |

### 3. Curly Coat Brushes

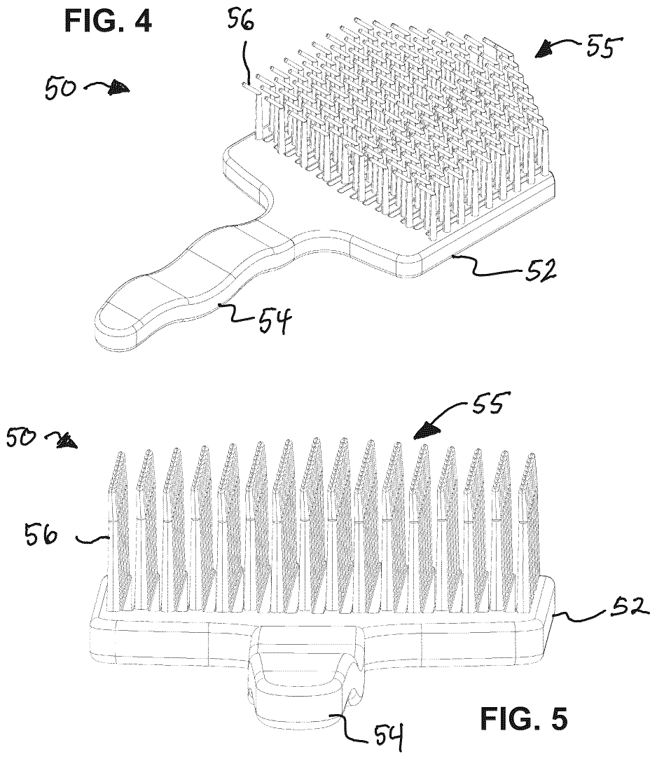

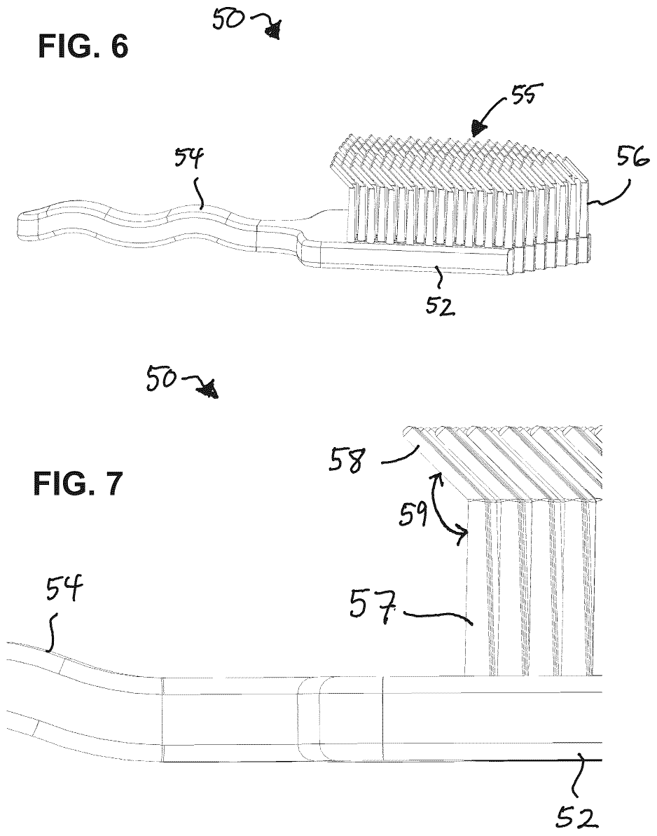

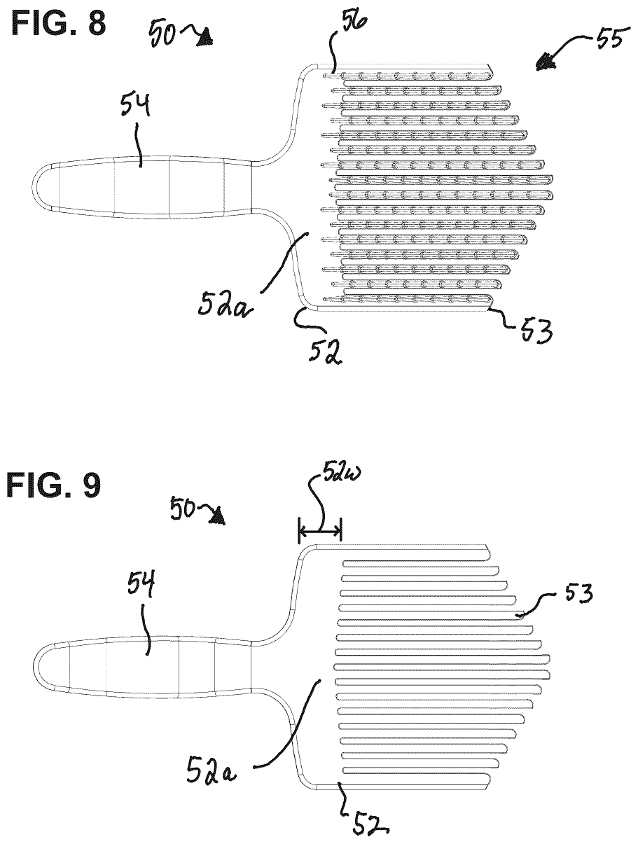

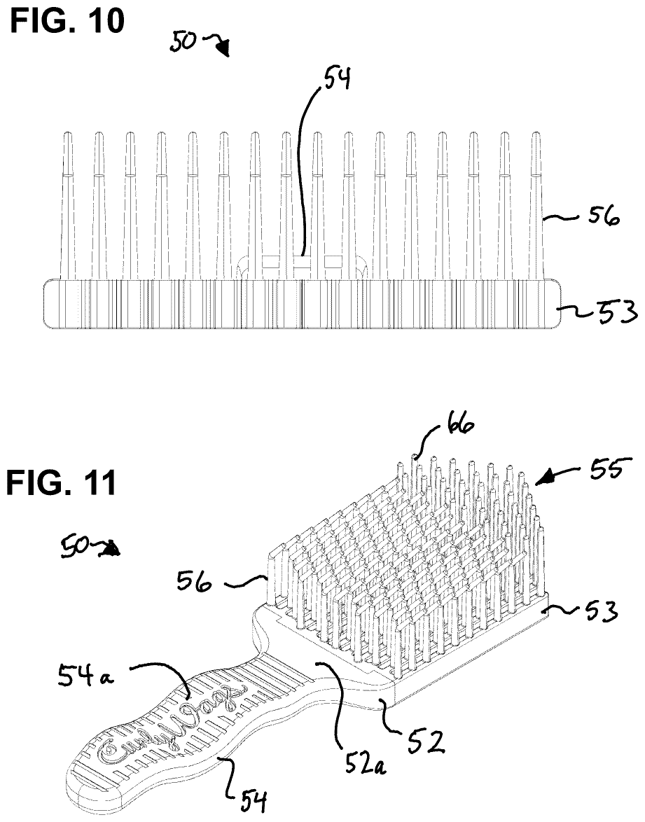

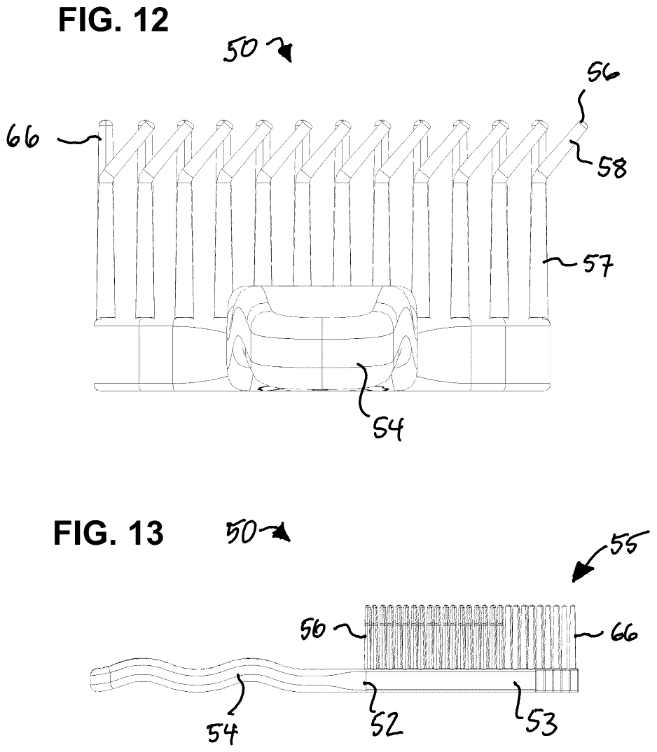

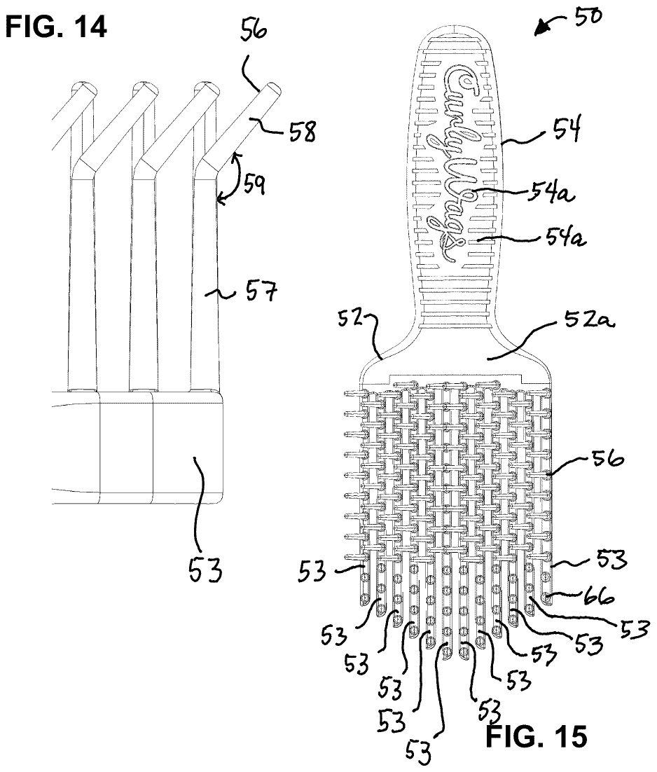

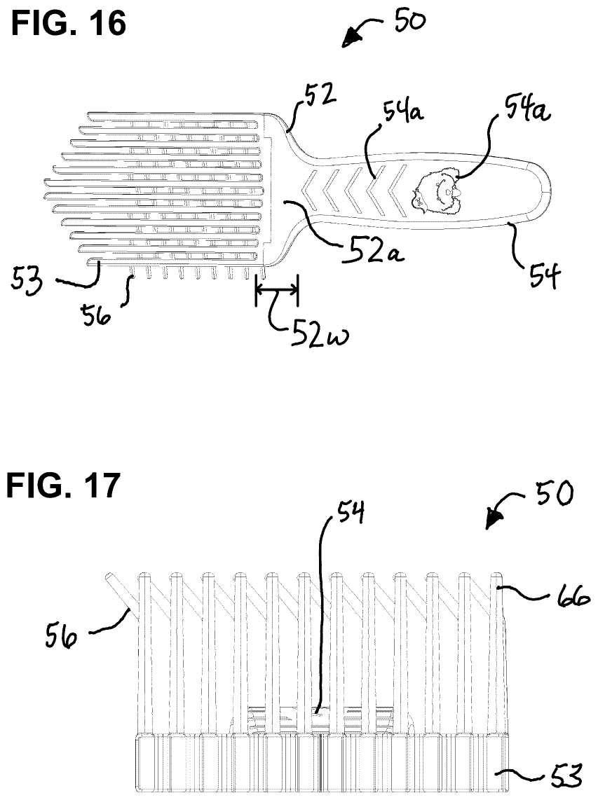

Referring now generally to FIGS. 4-20, the present disclosure also provides curly coat brushes 50 configured to groom a pet's curly coat. Generally, curly coat brushes 50 consistent with the present disclosure include a base 52, a handle 54 that extends outwardly from the base 52, and a brush head area 55 that includes a plurality of bristles 56,66, at least some of which are curly coat bristles 56. Although the curly coat brush 50 may be formed of any suitable material that is strong and not brittle, performance is most efficient and results are best when the outer surfaces of the curly coat brush 50 are smooth to avoid physically damaging the pet's hair/fur.

The base 52 secures the bristles 56,66. In some embodiments, the base 52 includes a plurality of base tines 53 extending outwardly from the base 52. When present, the plurality of base tines 53 increases the flexibility of the brush head area 55. Without wishing to be bound by theory, the inventors have observed that more flexible brush head area 55 configurations reduce the pet's discomfort while its coat is brushed, possibly due to a reduction in the frequency and/or magnitude with which one or more bristles 56,66 become snared in a knot or tangled portion of the pet's coat. In some embodiments, a curly coat brush 50 including a plurality of base tines 53 more closely mimics the grooming effects and experience achieved by using human fingers to detangle the pet's coat compared to a grooming brush that does not include a plurality of base tines 53. In some embodiments, removing loose pet hair from between the bristles 56,66 of a curly coat brush 50 that includes a plurality of base tines 53 is easier (e.g., faster or more efficient) than removing loose pet hair from between the bristles of a comparable grooming brush that does not include a plurality of base tines 53.

When base tines 53 are present, the base 52 may include a sturdy base portion 52a disposed between the base tines 53 and the handle 54. The sturdy base portion 52a provides structural support for the base tines 53 to reduce the likelihood that any one tine 53 will break away (e.g., snap off) the curly coat brush 50. The sturdy base portion 52a has a width 52w that may vary depending on, for example, the material(s) utilized in construction of the base 52 and the base tines 53, the thickness of the base 52, the dimensions (e.g., length, width, and thickness) of the base tines 53, the length of the bristles 56,66, and the characteristics of the pet breed(s) (e.g., length, thickness, and curliness of the pet breed's typical coat) for which the curly coat brush 50 is intended. In general, the width 52w must be greater when the base tines 53 are longer, narrower, and/or thinner; and/or the curly coat brush material is more brittle; and/or the pet breed's coat is typically curlier, thicker, and/or longer. In some embodiments, the width 52w is about 5 mm to about 50 mm, for example 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm.

In some embodiments, the base tines 53 extend outwardly from the base 52 in parallel with each other. In some embodiments, including those consistent with FIGS. 4-17, at least one base tine 53 is longer (e.g., extends from the base 52 farther) than at least one other base tine 53. In some embodiments, the plurality of base tines 53 extend outwardly from the base 52 to form a U-shaped or V-shaped pattern at the top end of the base 52. In some embodiments, including those consistent with FIGS. 4-17, the U-shaped or V-shaped pattern is convex (e.g., having base tines 53 at or near its center that extend farther from the base 52 than the base tines 53 extend from the outer edges of the base 52). In other embodiments, the U-shaped or V-shaped pattern is concave (e.g., having base tines 53 at or near its center that extend less from the base 52 than the base tines 53 extend from the outer edges of the base 52).

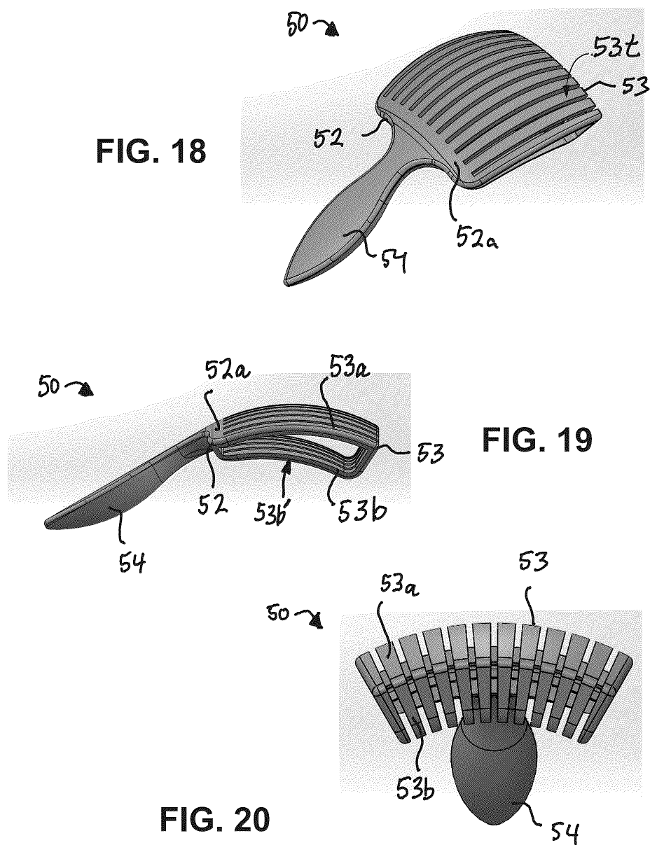

In some embodiments, such as the embodiment shown specifically in FIGS. 18-20, the base tines 53 extend outwardly from the base 52 such that the proximal ends of the base tines 53 form a straight line. In other embodiments, such as those shown specifically in FIGS. 4-17, the base tines extend outwardly from the base 52 such that the proximal ends of the base tines 53 form a curved or v-shaped pattern.

The handle 54 extends outwardly from the base 52 and is configured to fit comfortably in a user's hand. In some embodiments, the handle 54 extends outwardly from the base 52 in a direction opposite to the direction at which the base tines 53 extend from the base 52. In some embodiments, the handle 54 includes a curved profile, for example to increase ergonomics and reduce muscle stress and fatigue experienced by the user compared to similar brushes having a substantially straight handle. In some embodiments, the handle 54 includes a textured surface 54a, for example to increase a user's grip on the handle 54 while brushing a pet's coat with the curly coat brush 50.

The brush head area 55 includes a plurality of bristles 56,66, configured to pass through a collection of the pet coat's fur or hair. The plurality of bristles includes at least one curly coat bristle 56, and may also optionally include at least one straight bristle 66.

Each curly coat bristle 56 extends outwardly from the base 52 and/or from a base tine 53 in a direction generally orthogonal to the direction at which the handle 54 extends away from the base 52. Each curly coat bristle 56 has a smooth outer surface (e.g., does not include a texture or ridges) and includes a base portion 57 secured by the base 52 or a base tine 53, and a tip portion 58 that extends outwardly from a distal end of the base portion 57 at an angle 59. Without wishing to be bound by theory, it is currently believed that the angled tip portion 58 improves grooming of pet coats that have an undercoat and pet coats that have long hair types. In some embodiments, the angle 59 is obtuse. In some embodiments, the angle 59 is 91° to 179°, for example 91°, 92°, 93°, 94°, 95°, 96°, 97°, 98°, 99°, 100°, 101°, 102°, 103°, 104°, 105°, 106°, 107°, 108°, 109°, 110°, 111°, 112°, 113°, 114°, 115°, 116°, 117°, 118°, 119°, 120°, 121°, 122°, 123°, 124°, 125°, 126°, 127°, 128°, 129°, 130°, 131°, 132°, 133°, 134°, 135°, 136°, 137°, 138°, 139°, 140°, 141°, 142°, 143°, 144°, 145°, 146°, 147°, 148°, 149°, 150°, 151°, 152°, 153°, 154°, 155°, 156°, 157°, 158°, 159°, 160°, 161°, 162°, 163°, 164°, 165°, 166°, 167°, 168°, 169°, 170°, 171°, 172°, 173°, 174°, 175°, 176°, 177°, 178°, or 179°. In some embodiments, the angle 59 is obtuse. In some embodiments, the angle 59 is 100° to 170°. In some embodiments, the angle 59 is obtuse. In some embodiments, the angle 59 is 110° to 160°. In some embodiments, the angle 59 is obtuse. In some embodiments, the angle 59 is 120° to 150°. In some embodiments, the angle 59 is obtuse. In some embodiments, the angle 59 is 130° to 140°. In some embodiments, the angle 59 is obtuse. In some embodiments, the angle 59 is 135°.

In some embodiments, such as those consistent with FIGS. 4-17, the length of the base portion 57 of the curly coat bristle 56 is greater than the length of the tip portion 58. In other embodiments, the length of the base portion 57 of the curly coat bristle 56 is the same as the length of the tip portion 58. In still other embodiments, the length of the base portion 57 of the curly coat bristle 56 is less than the length of the tip portion 58.

In some embodiments, such as those generally consistent with FIGS. 4-10, the tip portion 58 extends outwardly from the base portion 57 in a direction generally towards the handle 54. In other embodiments, such as those generally consistent with FIGS. 11-17, the tip portion 58 extends outwardly from the base portion 57 in a direction generally orthogonal to the direction at which the handle 54 extends from the base 52.

The base portion 57 may have a diameter that is consistent along its length in some embodiments. In other embodiments, the base portion 57 has a larger diameter at its intersection with the base 52 (e.g., the base tines 53) than at its top, for example to reduce the likelihood that the bristle 56 will detach from (e.g., snap off of) the curly coat brush 50.

Each straight bristle 66, when present, extends outwardly from the base 52 and/or from a base tine 53 in a direction generally orthogonal to the direction at which the handle 54 extends away from the base 52. The straight bristles 66, when present, extend linearly (e.g., without substantial deflection from a straight line) from the base 52 or from a base tine 53. In some embodiments, each straight bristle 66, when present, extends from the base 52 or from a base tine 53 in the same direction that the base portion of each curly coat bristle 56 extends from the base 52 or from a base tine 53.

The straight bristles 66, when present, may have a diameter that is consistent along its length in some embodiments. In other embodiments, the straight bristles 66 each have a larger diameter at its intersection with the base 52 (e.g., the base tines 53) than at its top, for example to reduce the likelihood that the straight bristle 66 will detach from (e.g., snap off of) the curly coat brush 50.

In some embodiments, the curly coat brush 50 includes 50 to 200 curly coat bristles 56, such as 50 curly coat bristles 56, 51 curly coat bristles 56, 52 curly coat bristles 56, 53 curly coat bristles 56, 54 curly coat bristles 56, 55 curly coat bristles 56, 56 curly coat bristles 56, 57 curly coat bristles 56, 58 curly coat bristles 56, 59 curly coat bristles 56, 60 curly coat bristles 56, 61 curly coat bristles 56, 62 curly coat bristles 56, 63 curly coat bristles 56, 64 curly coat bristles 56, 65 curly coat bristles 56, 66 curly coat bristles 56, 67 curly coat bristles 56, 68 curly coat bristles 56, 69 curly coat bristles 56, 70 curly coat bristles 56, 71 curly coat bristles 56, 72 curly coat bristles 56, 73 curly coat bristles 56, 74 curly coat bristles 56, 75 curly coat bristles 56, 76 curly coat bristles 56, 77 curly coat bristles 56, 78 curly coat bristles 56, 79 curly coat bristles 56, 80 curly coat bristles 56, 81 curly coat bristles 56, 82 curly coat bristles 56, 83 curly coat bristles 56, 84 curly coat bristles 56, 85 curly coat bristles 56, 86 curly coat bristles 56, 87 curly coat bristles 56, 88 curly coat bristles 56, 89 curly coat bristles 56, 90 curly coat bristles 56, 91 curly coat bristles 56, 92 curly coat bristles 56, 93 curly coat bristles 56, 94 curly coat bristles 56, 95 curly coat bristles 56, 96 curly coat bristles 56, 97 curly coat bristles 56, 98 curly coat bristles 56, 99 curly coat bristles 56, 100 curly coat bristles 56, 101 curly coat bristles 56, 102 curly coat bristles 56, 103 curly coat bristles 56, 104 curly coat bristles 56, 105 curly coat bristles 56, 106 curly coat bristles 56, 107 curly coat bristles 56, 108 curly coat bristles 56, 109 curly coat bristles 56, 110 curly coat bristles 56, 111 curly coat bristles 56, 112 curly coat bristles 56, 113 curly coat bristles 56, 114 curly coat bristles 56, 115 curly coat bristles 56, 116 curly coat bristles 56, 117 curly coat bristles 56, 118 curly coat bristles 56, 119 curly coat bristles 56, 120 curly coat bristles 56, 121 curly coat bristles 56, 122 curly coat bristles 56, 123 curly coat bristles 56, 124 curly coat bristles 56, 125 curly coat bristles 56, 126 curly coat bristles 56, 127 curly coat bristles 56, 128 curly coat bristles 56, 129 curly coat bristles 56, 130 curly coat bristles 56, 131 curly coat bristles 56, 132 curly coat bristles 56, 133 curly coat bristles 56, 134 curly coat bristles 56, 135 curly coat bristles 56, 136 curly coat bristles 56, 137 curly coat bristles 56, 138 curly coat bristles 56, 139 curly coat bristles 56, 140 curly coat bristles 56, 141 curly coat bristles 56, 142 curly coat bristles 56, 143 curly coat bristles 56, 144 curly coat bristles 56, 145 curly coat bristles 56, 146 curly coat bristles 56, 147 curly coat bristles 56, 148 curly coat bristles 56, 149 curly coat bristles 56, 150 curly coat bristles 56, 151 curly coat bristles 56, 152 curly coat bristles 56, 153 curly coat bristles 56, 154 curly coat bristles 56, 155 curly coat bristles 56, 156 curly coat bristles 56, 157 curly coat bristles 56, 158 curly coat bristles 56, 159 curly coat bristles 56, 160 curly coat bristles 56, 161 curly coat bristles 56, 162 curly coat bristles 56, 163 curly coat bristles 56, 164 curly coat bristles 56, 165 curly coat bristles 56, 166 curly coat bristles 56, 167 curly coat bristles 56, 168 curly coat bristles 56, 169 curly coat bristles 56, 170 curly coat bristles 56, 171 curly coat bristles 56, 172 curly coat bristles 56, 173 curly coat bristles 56, 174 curly coat bristles 56, 175 curly coat bristles 56, 176 curly coat bristles 56, 177 curly coat bristles 56, 178 curly coat bristles 56, 179 curly coat bristles 56, 180 curly coat bristles 56, 181 curly coat bristles 56, 182 curly coat bristles 56, 183 curly coat bristles 56, 184 curly coat bristles 56, 185 curly coat bristles 56, 186 curly coat bristles 56, 187 curly coat bristles 56, 188 curly coat bristles 56, 189 curly coat bristles 56, 190 curly coat bristles 56, 191 curly coat bristles 56, 192 curly coat bristles 56, 193 curly coat bristles 56, 194 curly coat bristles 56, 195 curly coat bristles 56, 196 curly coat bristles 56, 197 curly coat bristles 56, 198 curly coat bristles 56, 199 curly coat bristles 56, or 200 curly coat bristles 56.

In embodiments that include a plurality of straight bristles 66, the curly coat brush 50 includes 10 to 200 straight bristles 66, such as 10 straight bristles 66, 11 straight bristles 66, 12 straight bristles 66, 13 straight bristles 66, 14 straight bristles 66, 15 straight bristles 66, 16 straight bristles 66, 17 straight bristles 66, 18 straight bristles 66, 19 straight bristles 66, 20 straight bristles 66, 21 straight bristles 66, 22 straight bristles 66, 23 straight bristles 66, 24 straight bristles 66, 25 straight bristles 66, 26 straight bristles 66, 27 straight bristles 66, 28 straight bristles 66, 29 straight bristles 66, 30 straight bristles 66, 31 straight bristles 66, 32 straight bristles 66, 33 straight bristles 66, 34 straight bristles 66, 35 straight bristles 66, 36 straight bristles 66, 37 straight bristles 66, 38 straight bristles 66, 39 straight bristles 66, 40 straight bristles 66, 41 straight bristles 66, 42 straight bristles 66, 43 straight bristles 66, 44 straight bristles 66, 45 straight bristles 66, 46 straight bristles 66, 47 straight bristles 66, 48 straight bristles 66, 49 straight bristles 66, 50 straight bristles 66, straight bristles 66, 51 straight bristles 66, 52 straight bristles 66, 53 straight bristles 66, 54 straight bristles 66, 55 straight bristles 66, 56 straight bristles 66, 57 straight bristles 66, 58 straight bristles 66, 59 straight bristles 66, 60 straight bristles 66, 61 straight bristles 66, 62 straight bristles 66, 63 straight bristles 66, 64 straight bristles 66, 65 straight bristles 66, 66 straight bristles 66, 67 straight bristles 66, 68 straight bristles 66, 69 straight bristles 66, 70 straight bristles 66, 71 straight bristles 66, 72 straight bristles 66, 73 straight bristles 66, 74 straight bristles 66, 75 straight bristles 66, 76 straight bristles 66, 77 straight bristles 66, 78 straight bristles 66, 79 straight bristles 66, 80 straight bristles 66, 81 straight bristles 66, 82 straight bristles 66, 83 straight bristles 66, 84 straight bristles 66, 85 straight bristles 66, 86 straight bristles 66, 87 straight bristles 66, 88 straight bristles 66, 89 straight bristles 66, 90 straight bristles 66, 91 straight bristles 66, 92 straight bristles 66, 93 straight bristles 66, 94 straight bristles 66, 95 straight bristles 66, 96 straight bristles 66, 97 straight bristles 66, 98 straight bristles 66, 99 straight bristles 66, 100 straight bristles 66, 101 straight bristles 66, 102 straight bristles 66, 103 straight bristles 66, 104 straight bristles 66, 105 straight bristles 66, 106 straight bristles 66, 107 straight bristles 66, 108 straight bristles 66, 109 straight bristles 66, 110 straight bristles 66, 111 straight bristles 66, 112 straight bristles 66, 113 straight bristles 66, 114 straight bristles 66, 115 straight bristles 66, 116 straight bristles 66, 117 straight bristles 66, 118 straight bristles 66, 119 straight bristles 66, 120 straight bristles 66, 121 straight bristles 66, 122 straight bristles 66, 123 straight bristles 66, 124 straight bristles 66, 125 straight bristles 66, 126 straight bristles 66, 127 straight bristles 66, 128 straight bristles 66, 129 straight bristles 66, 130 straight bristles 66, 131 straight bristles 66, 132 straight bristles 66, 133 straight bristles 66, 134 straight bristles 66, 135 straight bristles 66, 136 straight bristles 66, 137 straight bristles 66, 138 straight bristles 66, 139 straight bristles 66, 140 straight bristles 66, 141 straight bristles 66, 142 straight bristles 66, 143 straight bristles 66, 144 straight bristles 66, 145 straight bristles 66, 146 straight bristles 66, 147 straight bristles 66, 148 straight bristles 66, 149 straight bristles 66, 150 straight bristles 66, 151 straight bristles 66, 152 straight bristles 66, 153 straight bristles 66, 154 straight bristles 66, 155 straight bristles 66, 156 straight bristles 66, 157 straight bristles 66, 158 straight bristles 66, 159 straight bristles 66, 160 straight bristles 66, 161 straight bristles 66, 162 straight bristles 66, 163 straight bristles 66, 164 straight bristles 66, 165 straight bristles 66, 166 straight bristles 66, 167 straight bristles 66, 168 straight bristles 66, 169 straight bristles 66, 170 straight bristles 66, 171 straight bristles 66, 172 straight bristles 66, 173 straight bristles 66, 174 straight bristles 66, 175 straight bristles 66, 176 straight bristles 66, 177 straight bristles 66, 178 straight bristles 66, 179 straight bristles 66, 180 straight bristles 66, 181 straight bristles 66, 182 straight bristles 66, 183 straight bristles 66, 184 straight bristles 66, 185 straight bristles 66, 186 straight bristles 66, 187 straight bristles 66, 188 straight bristles 66, 189 straight bristles 66, 190 straight bristles 66, 191 straight bristles 66, 192 straight bristles 66, 193 straight bristles 66, 194 straight bristles 66, 195 straight bristles 66, 196 straight bristles 66, 197 straight bristles 66, 198 straight bristles 66, 199 straight bristles 66, or 200 straight bristles 66.

In some embodiments, the bristles 56,66 are disposed evenly across the brush head area 55, for example to provide efficient and even grooming results. Spacing between adjacent bristles 56,66 may vary depending, for example, on the typical coat characteristics associated with the breed(s) of pet for which the curly coat brush 50 is intended. For example and without limitation, a curly coat brush 50 intended for grooming a pet breed typically having long, thick, and/or excessively curly coats may have relatively larger spacing between adjacent bristles 56,66 than a curly coat brush 50 intended for grooming a pet breed typically having shorter, thinner, and/or less curly coats.

In some embodiments, such as those consistent with FIGS. 4-17, the curly coat bristles 56 secured by a first base tine 53 are offset horizontally from the curly coat bristles 56 secured by a second adjacent base tine 53. In other embodiments, the curly coat bristles 56 secured by a first base tine 53 are horizontally aligned with the curly coat bristles 56 secured by a second adjacent base tine 53.

In some embodiments that include a plurality of straight bristles 66, such as those consistent with FIGS. 11-17, the straight bristles 66 secured by a first base tine 53 are offset horizontally from the straight bristles 66 secured by a second adjacent base tine 53. In other embodiments, the straight bristles 66 secured by a first base tine 53 are horizontally aligned with the straight bristles 66 secured by a second adjacent base tine 53.

In some embodiments that include a plurality of straight bristles 66, such as those consistent with FIGS. 11-17, the straight bristles 66 are disposed at the distal end of the curly coat brush 50 (e.g., away from the handle 54), while the curly coat bristles 56 are disposed between the straight bristles 66 and the handle 54. In other embodiments, the curly coat bristles 56 are disposed at the distal end of the curly coat brush (e.g., away from the handle 54), while the straight bristles 66 are disposed between the curly coat bristles 56 and the handle 54. In still other embodiments, the straight bristles 66 and the curly coat bristles 66 are disposed in an intermixed manner across the base 52.

Referring now specifically to FIGS. 18-20, a curly coat brush 50 consistent with one embodiment of the present disclosure includes a handle portion 54 generally consistent with the handle portions 54 described above relative to FIGS. 4-17, and a base 52 configured to support a plurality of bristles 56,66 (not shown for clarity). The base 52 includes a sturdy base portion 52a from which a plurality of base tines 53 extend generally away from the handle portion 54. The base tines 53 each have a looped shape generally including a top loop portion 53a on which the bristles 56,66 are disposed (not shown for clarity) and a bottom loop portion 53b connecting the distal end of the top loop portion 53a to the sturdy base portion 52a. In some embodiments, the bristles 56,66 extend generally orthogonally from the top surface 53t of the top loop portion 53a. The shape and dimensions of the bottom loop portion 53b influence the flexibility of the base tines 53. For example and without limitation, the bottom loop 53b of the base tines 53 shown in FIGS. 18-20 each include a concave arch 53b′ that increases the flexibility of the base tines 53 compared to similarly sized and dimensioned base tines 53 having a bottom loop 53b that includes a convex arch instead of the concave arch 53b′.

1. A method of grooming a curly coat of a pet, the method comprising:
shampooing the pet's coat;
conditioning the pet's coat;
brushing the pet's coat;
drying the pet's coat until it remains damp;
applying a curl cream to the pet's coat;
gently blow drying the pet's coat; and
waiting at least two weeks before shampooing, conditioning, or brushing the pet's coat.

2. (canceled)

3. (canceled)

4. The method of claim 1, wherein the step of conditioning the pet's coat comprises brushing conditioner into the pet's coat using a curly coat brush.

5. The method of claim 4, wherein the curly coat brush comprises:
a base; and
a plurality of curly coat bristles, each curly coat bristle comprising:
a base portion extending from the base, and
a plurality of bristles including a tip portion that extends at an obtuse angle from the base portion.

6. The method of claim 5, wherein the curly coat brush further comprises a plurality of straight bristles each extending linearly from the base.

7. (canceled)

8. The method of claim 1, wherein the step of brushing the pet's coat comprises brushing the pet's coat using a layer brushing technique that comprises:
parting the pet's coat using a comb or fingers to form a parted valley,
patting the coat at the parted area using a slicker brush or a curly coat brush,
pulling the slicker brush or the curly coat brush through a top layer of fur/hair in a direction of the fur/hair's growth while lifting the slicker brush or the curly coat brush away from the pet, releasing a small portion of the parted hair into the parted valley, and
repeating the steps of patting and pulling the slicker brush or the curly coat brush and releasing a small portion of the parted hair until the pet's entire coat has been treated.

9. (canceled)

10. The method of claim 1, wherein the step of applying curl cream to the pet's coat comprises:
(i) dispensing a small dollop of curl cream into a user's hand and covering both palms and all fingers of the user's hands by rubbing them together;
(ii) spreading the curl cream on a section of the pet's coat using a raking motion that directs the fur/hair away from the pet's body at a 90° angle;
(iii) ensuring that the curl cream is applied all the way to the base of the pet's fur/hair throughout the section of the pet's coat; and
(iv) repeating steps (i)-(iii) until all sections of the pet's coat have been thoroughly treated with curl cream.

11. The method of claim 1, wherein the step of waiting at least two weeks comprises:
optionally, after the pet's coat becomes saturated with water, drying the pet's coat to dampness;
applying, after the pet's coat becomes saturated with water, a curl spray to the pet's coat; and
gently blow drying the pet's coat.

12. The method of claim 1, wherein the step of waiting at least two weeks comprises applying a curl spray to the pet's coat.

13. The method of claim 1, wherein the method does not include rinsing the conditioner from the pet's coat before the step of drying the pet's coat until it remains damp.

14. A curl cream or spray for establishing or maintaining a curly pet coat, the curl cream or spray comprising:
a first conditioner;
a second conditioner;
a humectant;
an emollient;
a perfume; and
a solvent.

15. The curl cream or spray of claim 14, wherein the curl cream or spray comprises:
0.5% to 4.5% of a first conditioner;
0.01% to 1.5% of a second conditioner;
0.001% to 6% of a humectant;
0.25% to 15% of an emollient;
0.01% to 1.5% of a perfume; and
balance solvent.

16. The curl cream or spray of claim 14, wherein the first conditioner is selected from the group consisting of: glycerin, linseed extract, marshmallow root extract, arginine (base), silk amino acids, and guar hyroxypropyltrimonium chloride.

17. The curl cream or spray of claim 14, wherein the second conditioner is different from the first conditioner and is selected from the group consisting of: glycerin, linseed extract, marshmallow root extract, arginine (base), silk amino acids, and guar hyroxypropyltrimonium chloride.

18. (canceled)

19. (canceled)

20. (canceled)

21. (canceled)

22. The curl cream or spray of claim 14 further comprising 0.1% to 5% of a film-forming agent, wherein the film-forming agent is optionally polyquaternium 11.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A curly coat brush for grooming a pet with a curly coat, the curly coat brush comprising:
a base;
a handle extending outwardly from the base; and
a plurality of curly coat bristles, each curly coat bristle comprising:
a base portion extending from the base, and
a tip portion that extends at an obtuse angle from the base portion.

42. The curly coat brush of claim 41 further comprising a plurality of straight bristles each extending linearly from the base.

43. The curly coat brush of claim 42, wherein the straight bristles each extend from the base at an angle orthogonal to an angle at which the handle extends from the base.

44. (canceled)

45. (canceled)

46. (canceled)

47. (canceled)

48. (canceled)

49. (canceled)

50. (canceled)

51. (canceled)

52. (canceled)

53. (canceled)

54. The curly coat brush of claim 41, wherein the base comprises a plurality of base tines each extending away from the handle.

55. The curly coat brush of claim 54, wherein each base tine supports a plurality of curly coat bristles.

56. The curly coat brush of claim 54, wherein each base tine supports a plurality of straight bristles.

57. (canceled)

58. (canceled)
